(19) United States
(12) Patent Application Publication
(10) Pub. No.: US 2012/0123113 A1
(43) Pub. Date: May 17, 2012

(54) DI-SUBSTITUTED PYRIDINUM POLYMERS AND SYNTHESIS THEREOF

(75) Inventors: Marcel Jaspars, Aberdeenshire (GB); Wael Houssen, Aberdeenshire (GB); Zhibao Lu, Aberdeenshire (GB); Roderick Scott, Aberdeenshire (GB); RuAngelie Edrada-ebel, Strathclyde (GB); Ines Mancini, Povo-Trento (IT)

(73) Assignee: University Court of the University of Aberdeen, Aberdeenshire (GB)

(21) Appl. No.: 13/146,566

(22) PCT Filed: Jan. 27, 2010
(86) PCT No.: PCT/GB2010/050124

§ 371 Date: Nov. 8, 2011

(30) Foreign Application Priority Data
Jan. 27, 2009 (GB) 0901330.1
Oct. 21, 2009 (GB) 0918510.9

(51) Int. Cl. C07D 487/22; C07D 401/14

(52) U.S. Cl. 540/472; 546/255

(57) ABSTRACT

A method of producing a di-substituted pyridinium polymer by microwave-assisted polymerisation of a 2, 3, or 4-substituted pyridine monomer of the formula NC₅R₄—R′—X, wherein R is selected from hydrogen, hydroxyl, and substituted or unsubstituted alkyl, alkoxy, aryl, alkaryl, aralkyl, and alkenyl groups, R′ is a linking group, and X is a leaving group. Using this method, di-substituted pyridinium polymer compositions may be obtained wherein at least 50% of the di- substituted pyridinium polymer chains in the composition have the same degree of polymerisation.

The present invention concerns di-substituted pyridinium polymers and the synthesis thereof. Specifically, the present invention concerns di-substituted alkylpyridinium polymers and the synthesis thereof. More specifically the present invention concerns oligomeric di-substituted alkylpyridinium salt polymers and the synthesis thereof.

Di-substituted alkylpyridinium compounds, in particular oligomeric 1,3-alkylpyridinium salt polymers (1,3-APS), are known to be produced by sponges, for example the Haploscerid genera such as Haliclona, Amphimedon and Callyspongia, as part of their chemical defences, and these compounds have potentially useful biological properties. Diverse biological activities have been identified for various 1,3-APS compositions, including cytotoxicity, neurotoxicity and inhibition of action potentials, stimulation of transmitter release, inhibition of K⁺ conductances, and anticholinesterase activity. At least some of these observed actions of 1,3-APS compositions relate to the pore forming or membrane lesion effects of these compounds, which properties may be useful, for example, in the transfection of cells with genetic material.

Naturally occurring 1,3-APS are produced as a cocktail of different 1,3-APS compounds. However, there are problems associated with the use of such a naturally occurring cocktail of 1,3-APS compounds which include for example: variability of supply, whereby different biological samples (ie. natural sponges) may produce different materials; sustainability of supply, whereby natural sponges are rare and produce little material; the use of the naturally occurring mixtures, whereby individual 1,3-APS compounds may show different biological properties but are difficult to isolate, due to the same basic structure and very similar molecular weights of different 1,3-APS compounds; and inability to change the chemical structure of the compounds, whereby it is difficult to study structure-activity relationships of 1,3-APS compounds and thereby to produce materials with defined properties and applications.

It is therefore desirable to be able to isolate different individual 1,3-APS compounds, for the use of specific and pure samples of 1,3-APS compounds having a narrow range of molecular weights and defined biological activity, and also in order to determine the different biological activities of a particular 1,3-APS compound, for example with regard to the effect of the degree of polymerisation, and length and rigidity of linking chains upon biological activity.

An alternative approach to isolating individual naturally occurring 1,3-APS compounds, and di-substituted alkylpyridinium compounds in general, is to synthesise these compounds by laboratory methods. Di-substituted alkylpyridinium compounds generally naturally occur as high molecular weight linear oligomers, with 30 to 100 monomer units, a molecular weight ranging from 1 KDa to greater than 25 KDa, and varying lengths of aliphatic chains linking the pyridine units.

Some previous studies have succeeded in producing linear 1,3-APS oligomers, but the methods employed meant that the product obtained consisted of a mixture of linear and cyclic oligomers with a wide range of molecular weights (see for example, Davies-Coleman, M. T.; Faulkner, D. J. J. Org. Chem. 1993, 58, 5925-5930). The approach used in these studies was to synthesize the 3-substituted pyridine monomer and introduce a good leaving group, for example bromide, at the end of the alkyl chain. This monomer was then refluxed to initiate polymerisation to give oligomers with a small number of monomer units, such as a maximum of 15, and a low molecular weight.

Alternative methods based on this strategy incorporate an ether linkage in the linking chain (Gil, L. et al Tetrahedron Lett. 1995, 36, 2059-2062), or form cyclic dimers (Morimoto, Y.; Yokoe, C. Tetrahedron Lett. 1997, 38, 8981-8984)or tetramers with short linking chains(Shinoda, S. et al, Chem. Commun. 1998, 181).

A further study for producing macrocyclic 1,3-APS compounds is disclosed in Kaiser, A. et al., J. Am. Chem. Soc. 1998, 120, 8026-8034, which involves the formation and subsequent reaction of an N-(2,4-dinitrobenzene)pyridinium salt (Zinke salt) to yield linear 1,3-APS compounds of up to 8 monomer units. This sequence can then be continued to give access to larger oligomers with a defined number of monomer units and this synthesis can be adapted to allow the introduction of variable linker units. However this method has certain disadvantages, in that it is relatively complex, and has only been shown to work for small macrocyclic oligomers, thus this method is not feasible to obtain the 30 to 100 monomer units as found in natural 1,3-APS polymers.

WO 2004/113299 provides a method for producing 1,3-APS oligomers and related compounds using a solid support.

The present invention seeks to provide a new reliable method for the synthesis of di-substituted pyridinium compounds, such as di-substituted pyridinium oligomers and polymers. Specifically, the present invention seeks to provide a new reliable method for the synthesis of 1,3-APS compounds which are closely analogous with naturally occurring 1,3-APS and which may be conveniently synthesised in a controllable fashion.

Moreover, the present invention seeks to provide a method which allows the straightforward formation of large di-substituted pyridinium polymers with high molecular weight and with a consistent degree of polymerisation. Consistency of molecular weight will be referred to herein as “monodispersity”.

According to an aspect of the present invention there is provided a method of producing a di-substituted-pyridinium polymer, the method comprising the steps of:

obtaining a 2, 3, or 4-substituted pyridine monomer of the formula NC₅R₄—R′—X, wherein R is selected from hydrogen, hydroxyl, and substituted or unsubstituted alkyl, alkoxy, aryl, alkaryl, aralkyl, and alkenyl groups, R′ is a linking group, and X is a leaving group;

and polymerising the 2, 3, or 4-substituted pyridine monomer by microwave-assisted polymerisation.

In this way, a method is provided whereby it is possible to prepare a more highly monodisperse higher molecular weight di-substituted-pyridinium polymer with a larger number of monomer units consistently, more controllably and sustainably than by previous prior art methods.

Moreover the above method using microwave-assisted polymerisation provides further advantages, whereby the overall degree of polymerisation can be more conveniently controlled, di-substituted-pyridinium polymers with different linker groups can be conveniently generated, and the resultant polymer is more highly monodisperse. This method is therefore generally applicable to make di-substituted-alkylpyridinium polymers, preferably 1,3-APS, with desired linker groups, degrees of polymerisation, and monodispersity.

Preferably, the pyridine monomer is a 3-substituted pyridine monomer.

In this way 1,3-APS may be obtained from the above method of the present invention.

Preferably, R′ is selected from an alkylene group, an alkenyl-containing group, an alkynyl-containing group, and a cyclopropanyl-containing group. Moreover R′ may be further functionalised, for example for addition of fluorescent groups.

More preferably, R′ is selected from a group —(CH₂)ₘ—, wherein m is an integer from 2 to 15, a group having from 2 to 15 carbon atoms containing one or more alkenyl groups, a group having from 2 to 15 carbon atoms containing one or more alkynyl groups, and cis- or trans- —(CH₂)ₚ-cyclopropanyl-(CH₂)_{q}— wherein p and q are the same or different and are integers from 1 to 6.

Thus, R′ groups include methylene, ethylene, propylene, butylene, pentylene, hexylene, heptylene, octylene, nonylene, decylene, undecylene and dodecylene. For example, R′ may be a propylene, heptylene, octylene or dodecylene group. In certain preferred embodiments, R′ is a docecylene group.

X may be a halide, triflate, mesylate or tosylate group. Preferably, X is bromide, chloride or iodide.

For example, the pyridine monomer may be 3-(3-chloropropyl)pyridine, 3-(7-bromoheptyl)pyridine, 3-(7-chloroheptyl)pyridine, 3-(8-bromooctyl)pyridine, 3-(12-bromododecyl)pyridine and 3-(12-chlorododecyl)pyridine.

Microwave-assisted polymerisation as discussed herein relates to the polymerisation of monomers as assisted by heating achieved through the exposure of the reacting species to microwave radiation. Thus, the present invention uses microwave radiation heating to assist the polymerisation of pyridine monomers to form di-substituted pyridinium polymers. In this respect, microwave radiation offers a number of advantages over conventional heating methods, such as noncontact heating, instantaneous and rapid heating, and highly specific heating.

Various microwave reactors suitable for performing the method of the present invention and the operation of these apparatus will be apparent to those skilled in the art. Such microwave reactors may for example include monomodal microwave reactors such as the Emrys Liberator (Biotage), CEM Discover BenchMate, Milestone Ethos TouchControl and Lambda MicroCure2100 BatchSystem.

The progress of the microwave assisted polymerisation may be monitored by means of ¹H-NMR in a CDCL₃/CD₃OD solvent, Matrix Assisted Laser Desorption Ionisation Time Of Flight Mass Spectrometry (MALDI-TOF-MS) or ElectroSpray Ionization Mass Spectrometry (ESI-MS).

Conveniently, the pyridine monomer may be dissolved in a solvent such as methanol prior to polymerisation.

The microwave-assisted polymerisation step may be carried out at a temperature between 100° C. and 200° C. For example, the microwave-assisted polymerisation step may be carried out at a temperature of from 120 to 150° C., such as 130° C.

The microwave-assisted polymerisation step may be carried out from between 20 minutes and 80 hours. Longer reaction times lead to greater degrees of polymerisation.

In this way, it has been found that the degree of polymerisation, polymer chain length and molecular weight of the di-substituted-pyridinium polymer product may be controlled by the duration of the polymerisation step.

The microwave-assisted polymerisation step may be carried out at a pressure of between 7 and 9 bar. For example, the microwave-assisted polymerisation step may be carried out at a pressure of 8 bar.

The microwave-assisted polymerisation step may be performed on a Biotage Initiator Microwave Synthesizer. Wherein the microwave-assisted polymerisation step may be carried out at a power of 30 to 40 Watts.

According to another aspect of the present invention there is provided a di-substituted pyridinium polymer obtained from the aforementioned method of the present invention.

The di-substituted pyridinium polymers obtained from the method of the present invention may be a linear or cyclic. Furthermore, linear polymers may be obtained from cyclic polymers which are obtained from the method of the present invention, for example, by ring opening of cyclic polymers.

According to yet another aspect of the present invention there is provided a di-substituted pyridinium polymer composition comprising polymer chains of the formula NC₅R₄—R′—[X⁻N⁺C₅R₄—R′—]ₙ—NC₅R₄—R′X or [X⁻N⁺C₅R₄—R′—]ₙ wherein R is selected from hydrogen, hydroxyl, and substituted or unsubstituted alkyl, alkoxy, aryl, alkaryl, aralkyl, and alkenyl groups, R′ is a linking group, X is a counter ion, n is the degree of polymerisation and is between 40 and 70, wherein at least 50% of the di-substituted pyridinium polymer chains in the composition have the same degree of polymerisation.

The compositions of the present invention show a higher monodispersity than in the prior art and in this way a more pure supply of a individual di-substituted-pyridinium polymer is obtained. The compositions of the present invention may thus have more clearly and narrowly defined properties and more potent biological activity.

Preferably, at least 55% of the di-substituted pyridinium polymer chains in the composition have the same degree of polymerisation.

In this way an even higher purity of an individual di-substituted-pyridinium polymer is obtained.

Preferably, n is 50 to 70, for example 51, 60 or 63.

Preferably, the di-substituted-pyridinium polymer comprises pyridinium rings substituted by R′ at the 3 position.

Preferably, R′ and X are defined as discussed above in connection with the method of the present invention.

Preferred di-substituted pyridinium polymer compositions of the present invention comprise poly-(3-(12-bromododecyl)pyridine of 50 to 60 monomer units and having a molecular weight of 12 to 15 kDa. Particularly preferred compositions comprise poly-(3-(12-bromododecyl)pyridine having 51 monomer units or poly-(3-(12-bromododecyl)pyridine having 60 monomer units.

Further preferred di-substituted pyridinium polymer compositions of the present invention comprise poly-(3-(8-bromooctyl)pyridine) of 60 to 70 monomer units and having a molecular weight of 11 to 13 kDa. Particularly preferred compositions comprise poly-(3-(8-bromooctyl)pyridine) having 63 monomer units.

The di-substituted pyridinium polymer composition of the present invention may have various biological activities including antibacterial activity such as against E. coli and S. aureus, haemolytic activity, increasing neurotransmission such as by acetylcholine esterase inhibition, and cell pore formation such as for use in cell transfection, for example of DNA into a cell.

Accordingly the di-substituted pyridinium polymer composition of the present invention may be used as an antibacterial agent, an acetylcholine esterase inhibitor, a haemolytic agent, or a transfection reagent.

Definitions

The following terms and abbreviations will be used throughout the specification and are intended to be construed in accordance with the following definitions.
APS: alkylpyridinium salt
poly-APS: alkylpyridinium salt polymers
polyAPS3-Cl: poly-(3-(3-chloropropyl)pyridine)
polyAPS7-Br: poly-(3-(7-bromoheptyl)pyridine)
polyAPS7-Cl: poly-(3-(7-chloroheptyl)pyridine)
polyAPS8-Br: poly-(3-(8-bromooctyl)pyridine)
polyAPS12-Br: poly-(3-(12-bromododecyl)pyridine)
polyAPS12-Cl: poly-(3-(12-chlorododecyl)pyridine)
AchE: Acetyl cholinesterase
GFP: Green fluorescent protein
HEK 293: Human embryonic kidney cell line
EMEM: Eagle's minimum essential medium
FCS: Foetal calf serum
ES: Embryonic stem cell
LIF: Leukaemia Inhibitory Factor
NEA: Non-essential amino acids
EB: Embryoid body
RA: Retinoic acid
MEF: Mouse embryonic fibroblast
EGF: Epidermal growth factor
bFGF: basic Fibroblast growth factor
FACS: Fluorescence activated cell sorting
MIC: Minimal inhibitory concentration

Embodiments of the present invention will now be discussed in detail with reference to the followind drawings, in which:

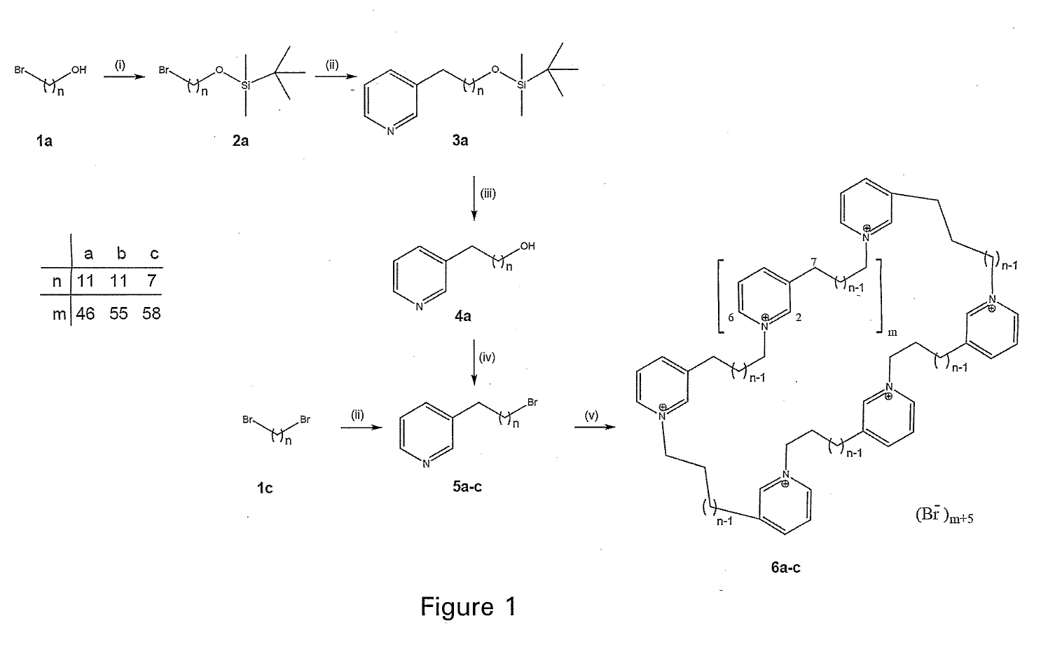

FIG. 1 shows an example synthesis of poly(1,3-alkylpyridinium) bromide salts, wherein the reagents, conditions and yields of the steps are: i) tert-butyldimethylsilyl chloride, triethylamine, 4-dimethyl aminopyridine, DCM, stiring overnight, 95%; ii) 3-picoline, diisopropylamine, THF, nBuLi, −78° C. to 0° C., stirring, 80% of 3a and 35% of 5c; iii) tetrabutylammonium fluoride, THF, 90%; iv) HBr, toluene, reflux overnight, 60% v) reflux in acetonitrile in the presence of Kl followed by microwave irradition either at 130° C./8 bar/40 w/30 min for compound 6a or at 130° C./8 bar/30 w/60 h for compound 6c.

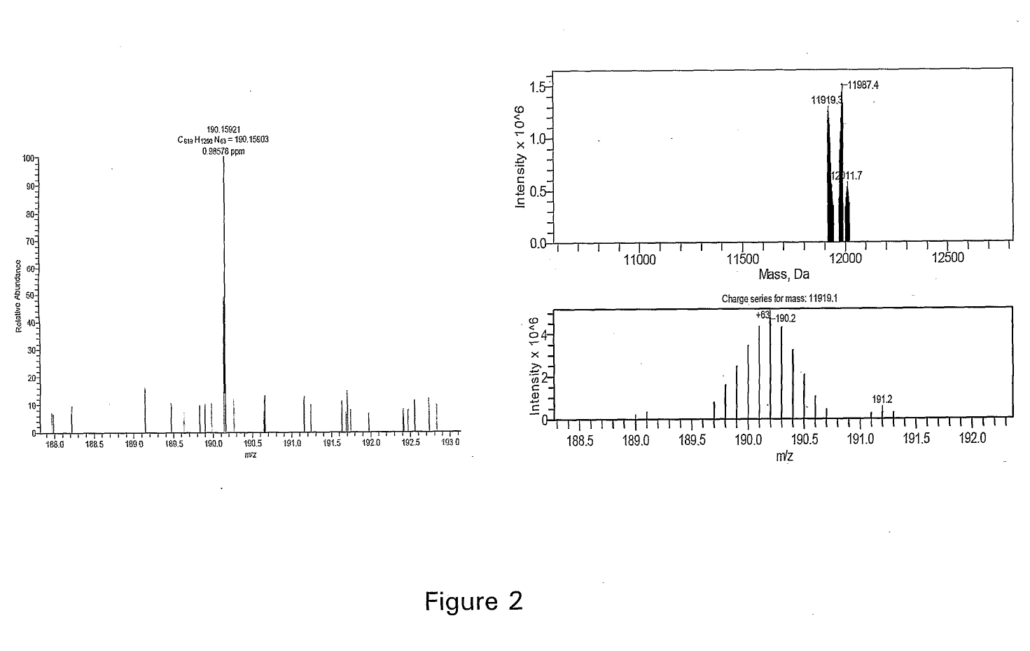

FIG. 2 shows mass spectra for polyAPS8-Br wherein the left panel shows a centroid mode spectrum for polyAPS8-Br at z=63 m/z 190.2 and the right panels show the deconvoluted MS spectra exhibiting 63 monomer units at 11980.0186 (11.9 kDa) for the polymer exhibiting a monomer unit of C₁₃H₂₀N as confirmed by HRFTMS data. The polymer is cyclic where the number of nitrogens is equivalent to the number of positive charges with no halogens.

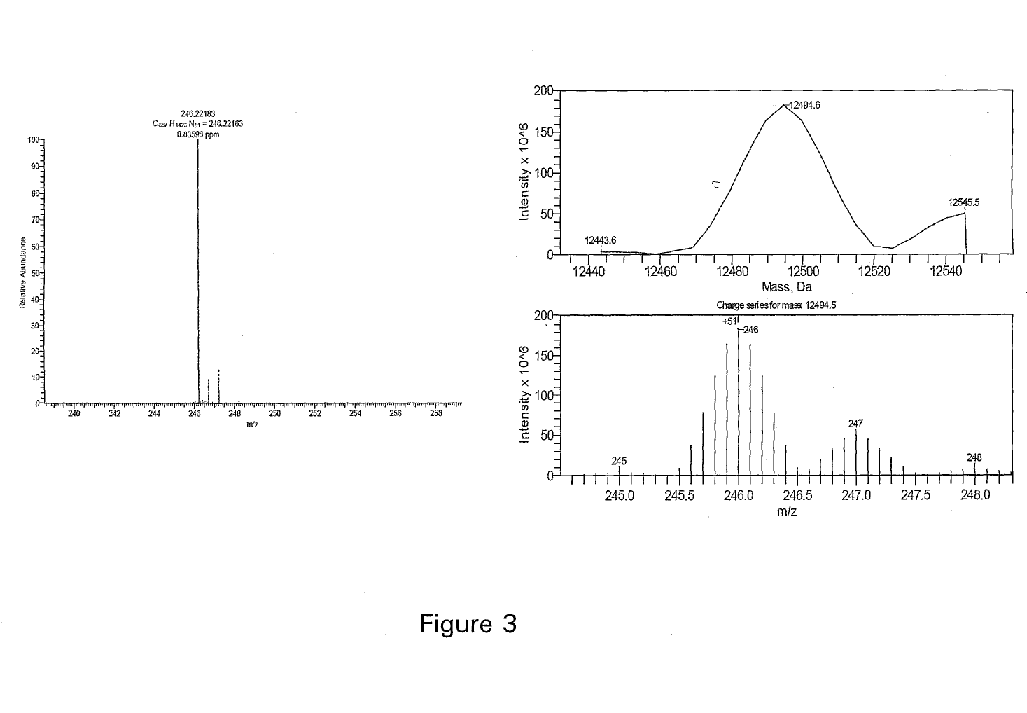

FIG. 3 shows mass spectra for polyAPS12-Br (APS12) wherein the left panel shows a centroid mode spectrum at z=51 m/z 246 and the right panels show the deconvoluted MS spectra exhibiting 51 monomer units at 12557.303 (12.5 kDa) for the polymer exhibiting a monomer unit of C₁₇H₂₈N as confirmed by HRFTMS data. The polymer is cyclic where the number of nitrogens is equivalent to the number of positive charges with no halogens.

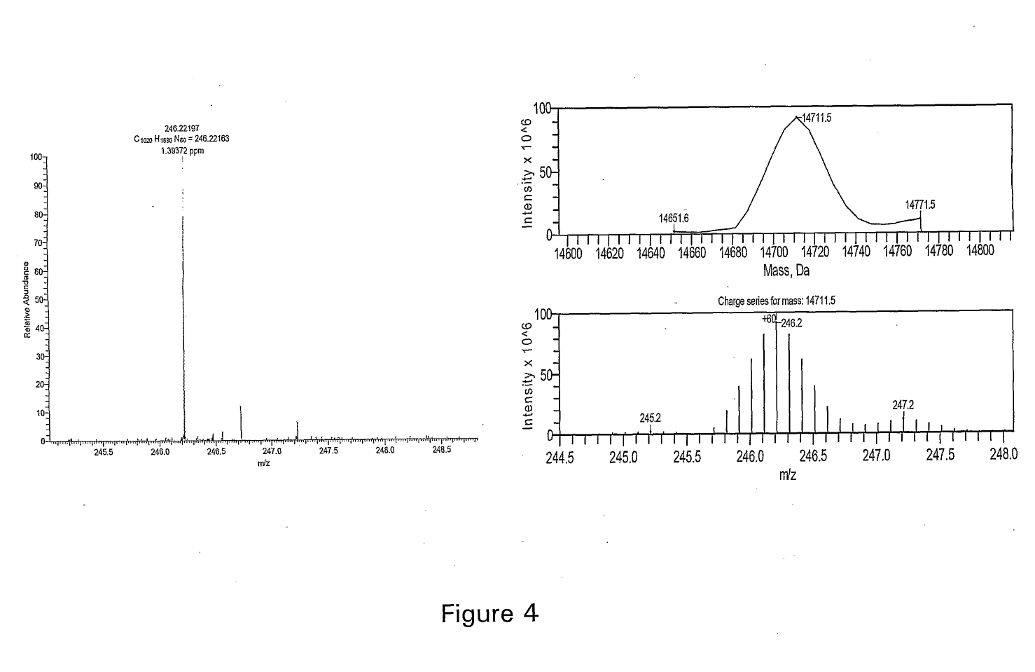

FIG. 4 shows mass spectra for polyAPS12-Br (APS12-2) wherein the left panel shows a centroid mode spectrum at z=60 m/z 246.2 and the right panels show the deconvoluted MS spectra exhibiting 60 monomer units at 14773.298 (14.7kDa) for the polymer exhibiting a monomer unit of C₁₇H₂₈N as confirmed by HRFTMS data. The polymer is cyclic where the number of nitrogens is equivalent to the number of positive charges with no halogens.

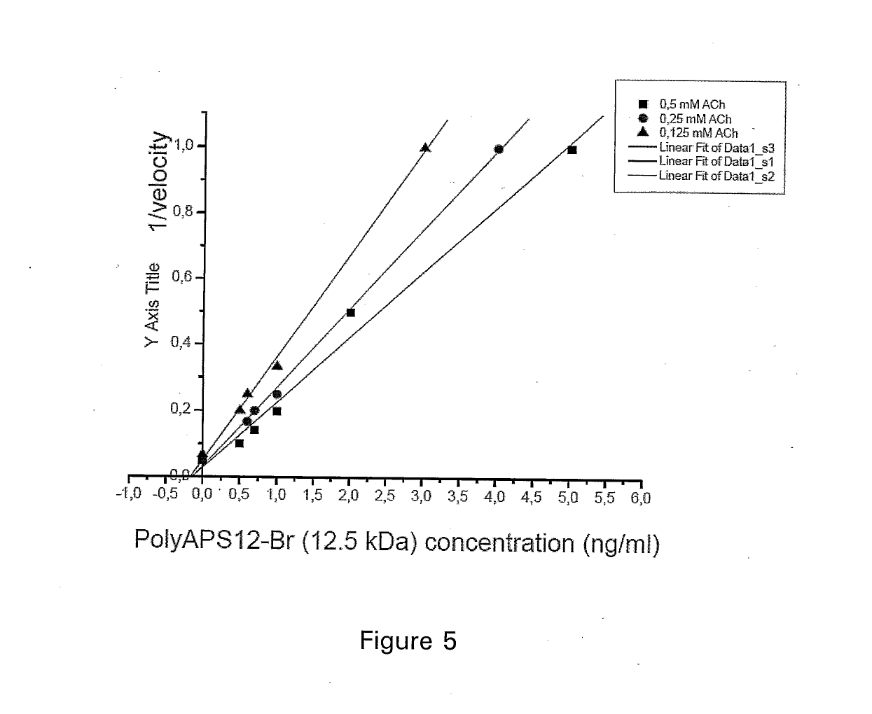

FIG. 5 is a Dixon plot of AChE inhibition for polyAPS12-Br (12.5 kDa), showing that synthetic analogues of poly-APS have the same spectrum of biological activity as natural poly-APS.

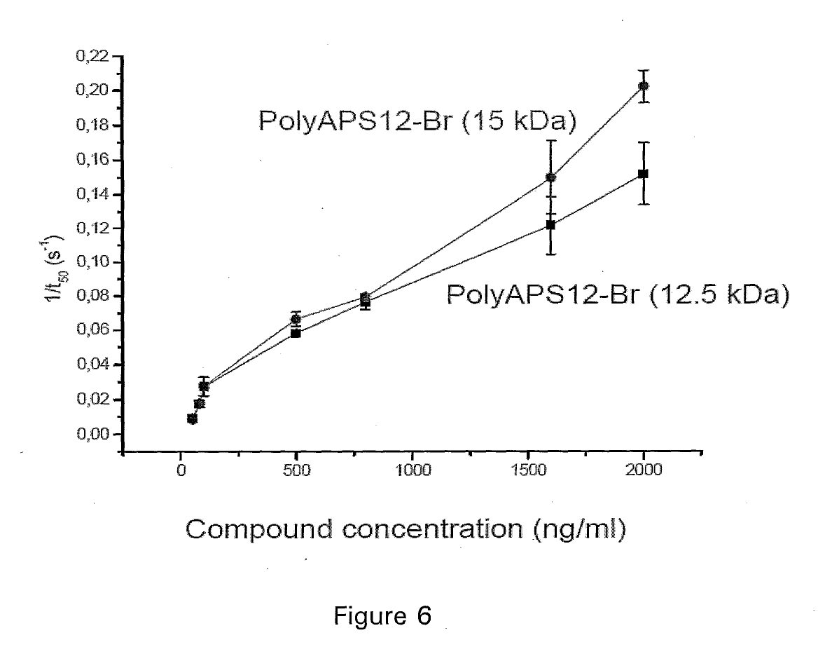

FIG. 6 is a plot of haemolysis activity of polyAPS12-Br (15 kDa) and PolyAPS12-Br (12.5 kDa), showing that synthetic analogues of poly-APS have the same spectrum of biological activity as natural poly-APS.

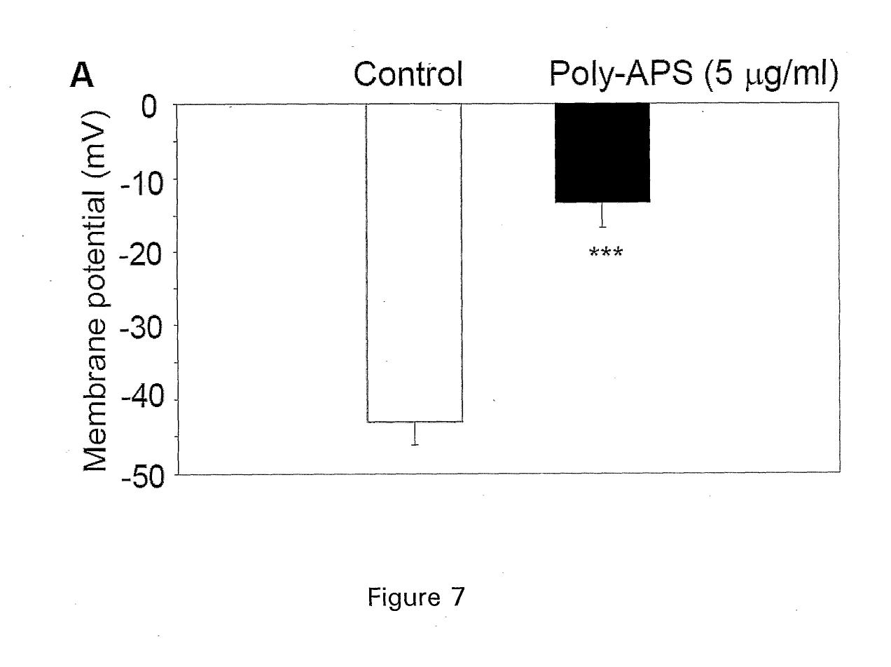

FIG. 7 is a bar chart showing the acute actions of natural poly-APS on the basic membrane properties of undifferentiated mouse embryonic stem cells, whereby reduction in membrane potential produced by 5 μg/mL poly-APS (n=6; P<0.005) is demonstrated.

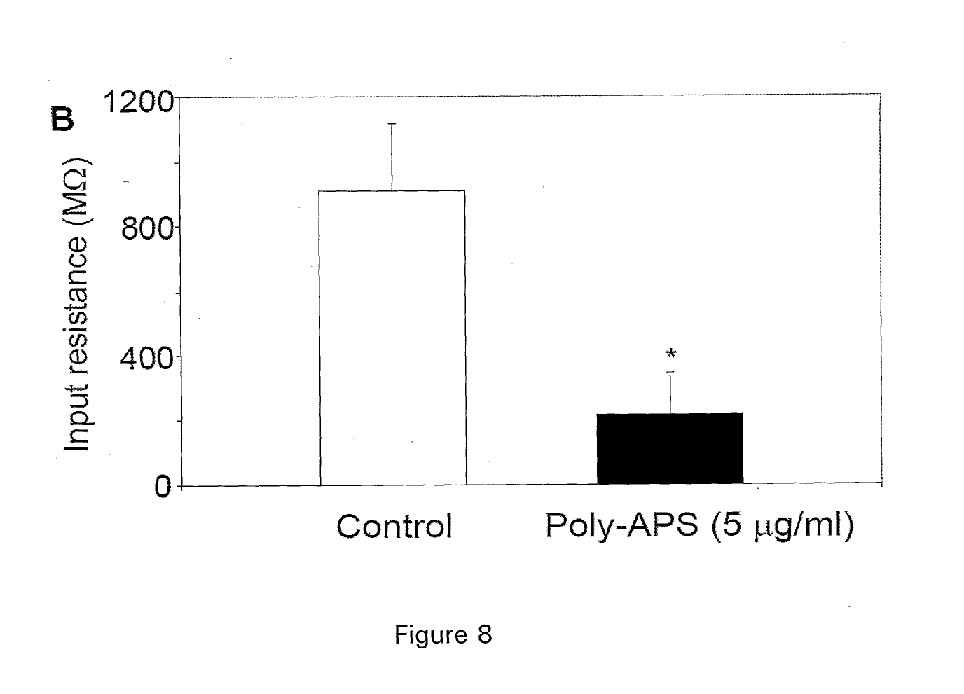

FIG. 8 is a bar chart showing the acute actions of natural poly-APS on the basic membrane properties of undifferentiated mouse embryonic stem cells, whereby the mean resting input resistance (n=8) and the input resistance after application of poly-APS (n=5; P<0.03) is demonstrated. Input resistance at −70 mV was obtained from electrotonic potentials evoked by −100 pA current step commands.

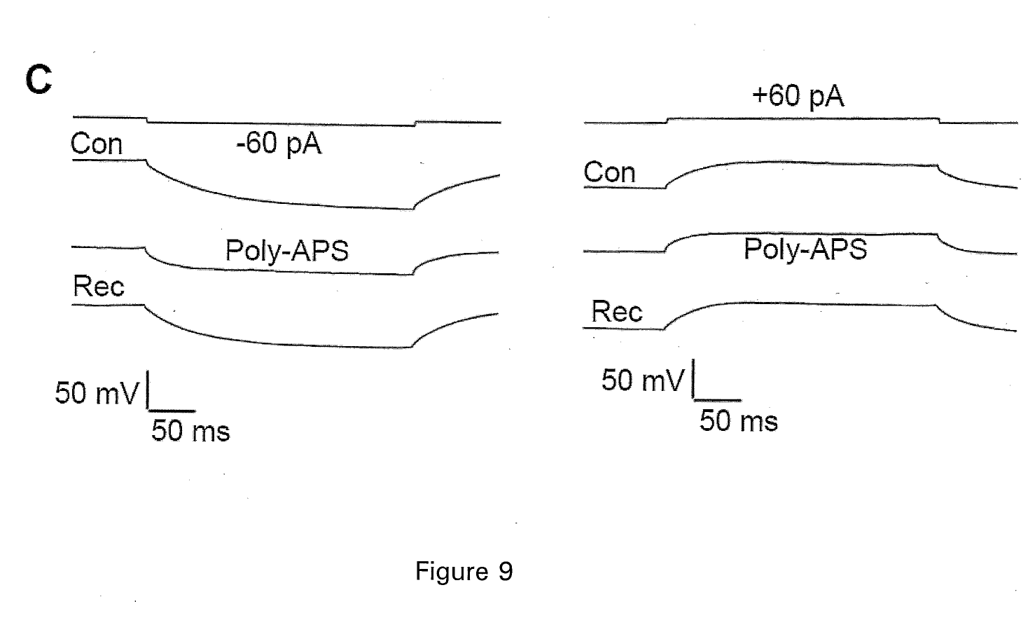

FIG. 9 shows example currents (±60 pA) and voltage traces recorded under control conditions (Con) in the presence of natural poly-APS and on recovery (Rec) for the acute actions of poly-APS on the basic membrane properties of undifferentiated mouse embryonic stem cells.

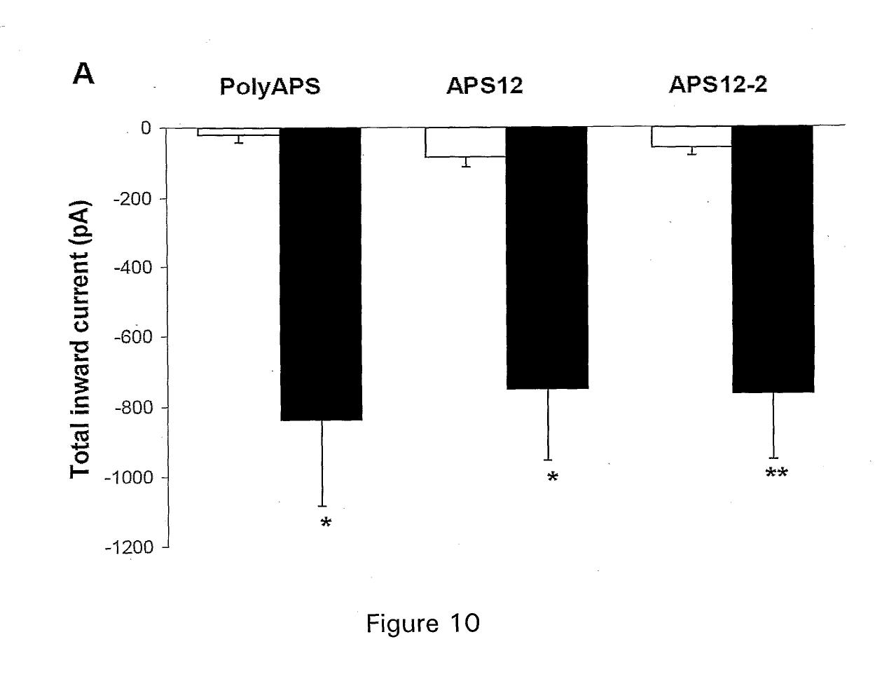

FIG. 10 is a bar chart showing that under voltage clamp poly-APS and its synthetic analogues, polyAPS12-Br (12.5 kDa; APS12) and polyAPS12-Br (15 kDa; APS12-2), evoked inward currents in undifferentiated mouse embryonic stem cells, wherein resting holding current at -70 mV and holding current after application of 5 μg/mL natural poly-APS (n=6; *P<0.02), 5 μg/mL polyAPS12-Br (15 kDa) (APS12-2; n=10; *P<0.01) and 5 μg/mL polyAPS12-Br (12.5 kDa) (APS12; n=10; **P<0.002) is shown.

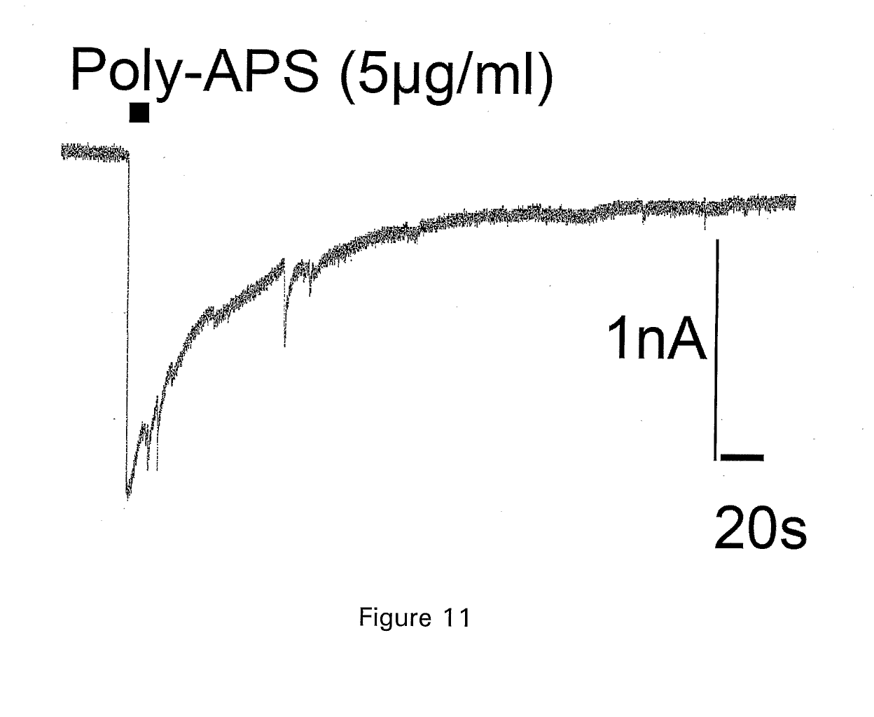

FIG. 11 is a current record showing a reversible inward current activated by natural poly-APS from a holding potential of −70 mV.

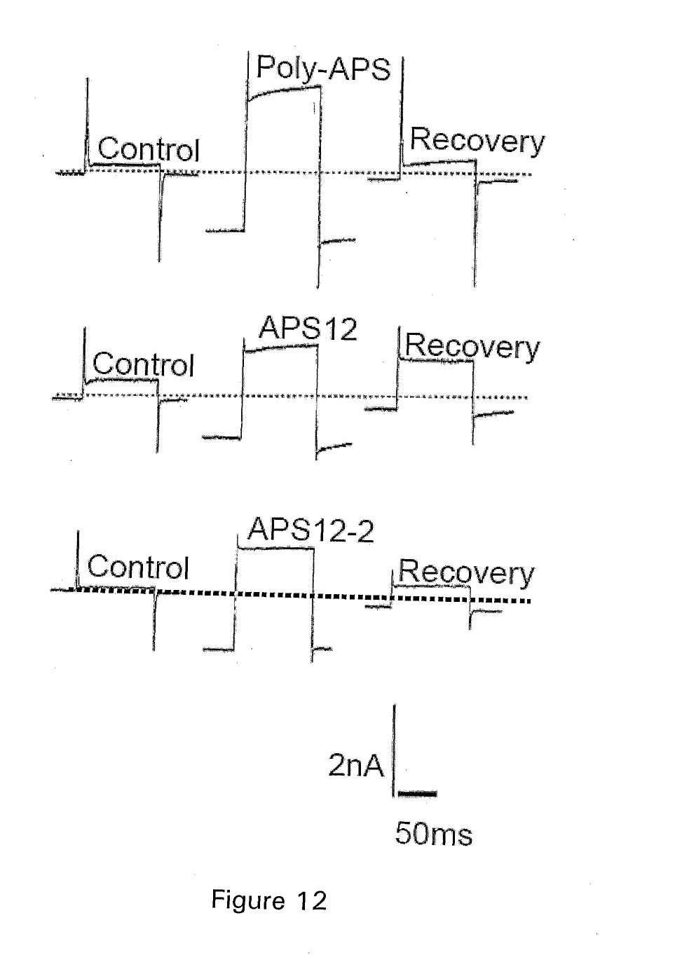

FIG. 12 shows example records of current responses to 100 ms voltage step commands of +130 mV (V_{c}+60 mV), under control conditions, during the peak drug response of natural poly-APS, polyAPS12-Br (12.5 kDa; APS12) and polyAPS12-Br (15 kDa; APS12-2) (all at 5 μg/mL) under voltage clamp and after 10-20 minutes recovery. The resting holding voltage was −70 mV and the dotted lines denote 0 pA.

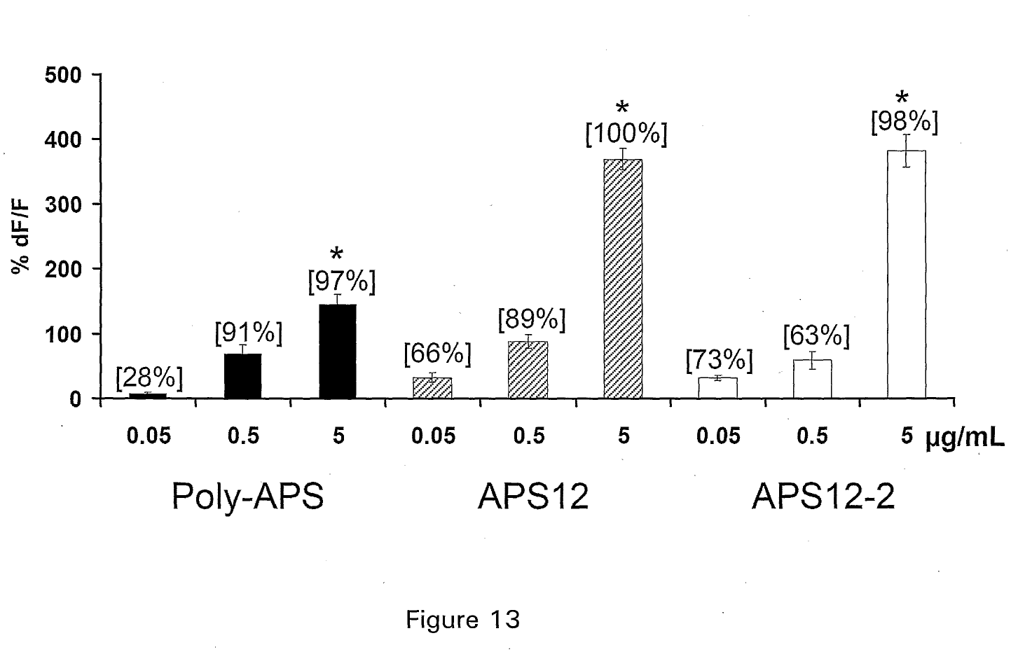

FIG. 13 is a bar chart showing that natural poly-APS and its synthetic analogues, polyAPS12-Br (12.5 kDa, APS12) and polyAPS12-Br (15 kDa; APS12-2), evoked Ca²⁺ transients measured using fura-2AM in undifferentiated mouse embryonic stem cells, wherein dose-response data is shown for the whole population of stem cells studied (responders and non-responders included) for natural poly-APS (n=67), polyAPS12-Br (15 kDa, APS12-2) (n=47) and polyAPS12-Br (12.5 kDa; APS12) (n=51); for each compound, 0.5 Vs 5 μg/mL, *P <0.0001. The values in square brackets give the percentage of cells responding to each dose.

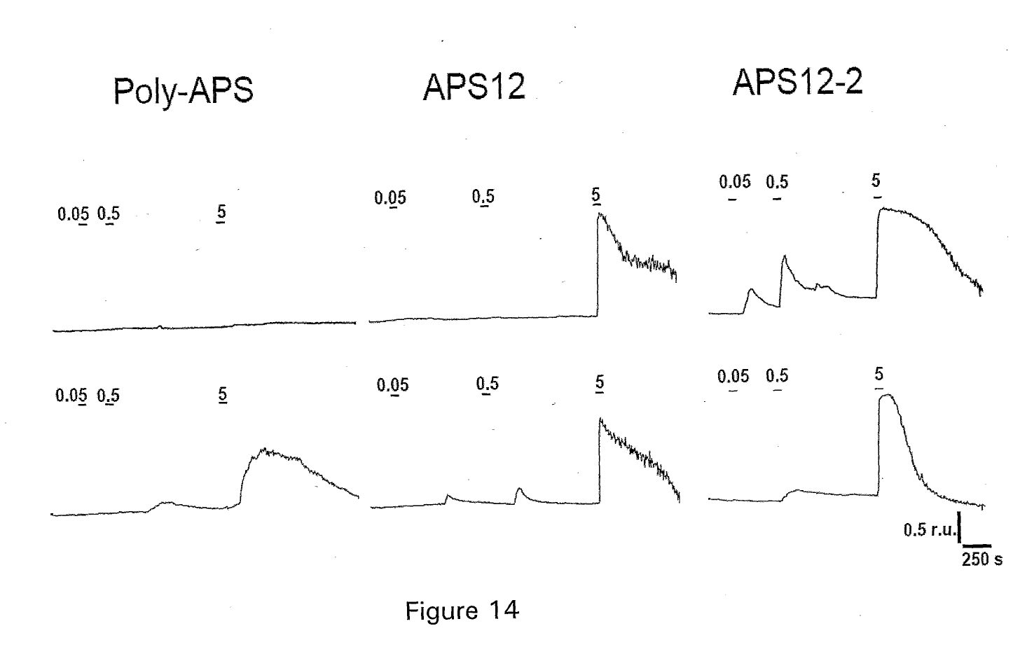

FIG. 14 shows that natural poly-APS and its synthetic analogues, polyAPS12-Br (12.5 kDa, APS12) and polyAPS12-Br (15 kDa; APS12-2), evoked Ca²⁺ transients measured using fura-2AM in undifferentiated mouse embryonic stem cells, wherein example traces show the variations in the changes in fluorescence ratio induced by poly-APS, polyAPS12-Br (12.5 kDa, APS12) and polyAPS12-Br (15 kDa, APS12-2).

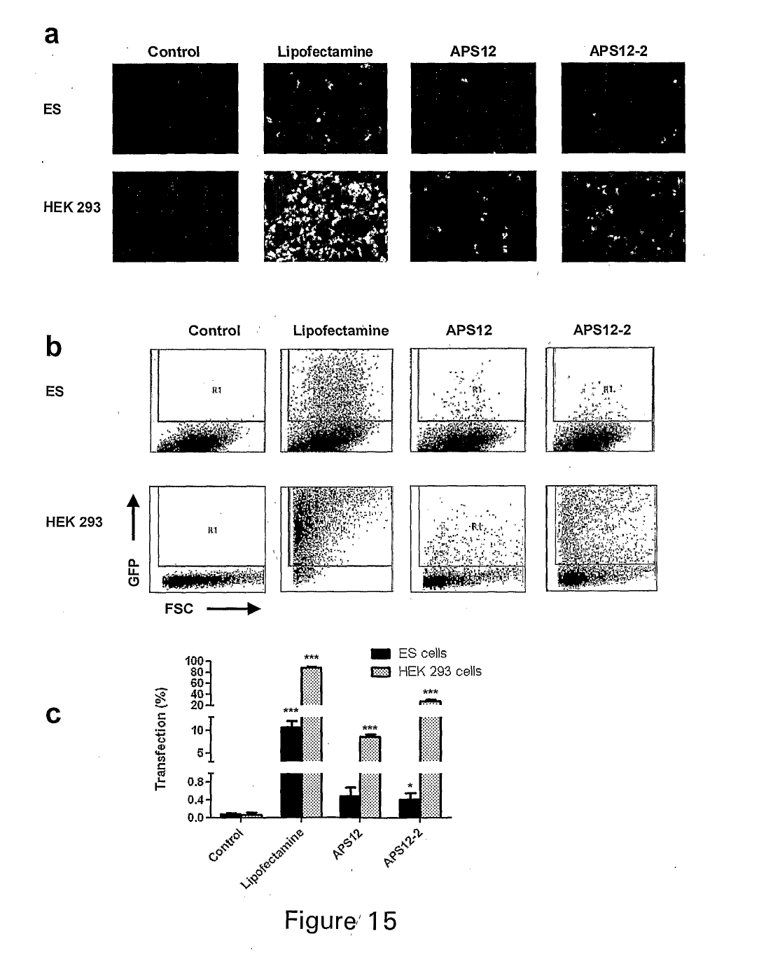

FIG. 15 shows the transient transfection of undifferentiated mouse ES and HEK 293 with pMAX-GFP using lipofectamine, APS12 and APS12-2, wherein panels A show confocal images of undifferentiated mouse ES cells and HEK 293 treated with no transfection vehicle (control), lipofectamine (2 mg/mL), APS12 (5 μg/mL) and APS12-2 (5 μg/mL) respectively in the presence of pMAX-GFP. Images were captured at 48 hours post-transfection and each is representative of five experimental repeats, and wherein representative flow cytometry dot plots B show populations (gated) of ES and HEK 293 that can express EGFP after 48 h tranfection using no transfection vehicle (control), lipofectamine (2 mg/mL), APS12 (5 μg/mL) and APS12-2 (5 μg/mL) respectively, and wherein bar charts C show percent transfection of mouse ES and HEK 293 cells as analysed by flow cytometry and data shown are the mean±SEM.

The following Examples describe syntheses of specific alkyl pyridium salt polymers (poly-APS) and their preceding intermediates. Experimental details are provided in the subsequent Experimental section.

### EXAMPLE 1

### Synthesis of poly-(3-(7-bromoheptyl)pyridine) (polyAPS7-Br)

PolyAPS7-Br was synthesised using microwave-assisted polymerisation of the monomer unit, 3-(7-bromoheptyl)pyridine in methanol (7) following Scheme 1. The monomer unit (7) was prepared from 1,6-hexanediol (1) by reflux with hydrobromic acid in toluene to give 6-bromo-l-hexanol (2), which was then protected by a silyl group. Coupling with 3-picoline followed by de-protection with TBNF gave 7-(3-pyridyl)heptanol (5), which could be transformed into the monomer, 3-(7-bromoheptyl)pyridine (7) by reflux with hydrobromic acid and neutralisation.

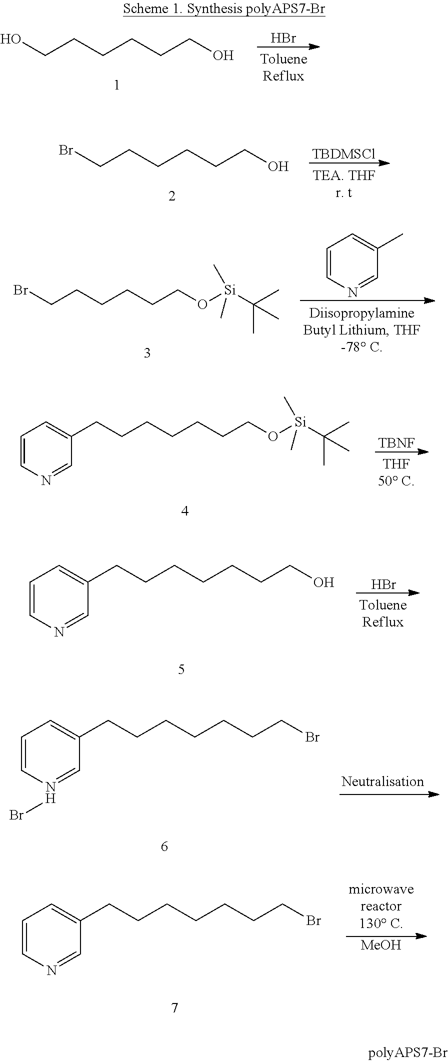

### EXAMPLE 2

### Synthesis of poly-(3-(7-chloroheptyl)pyridine) (polyAPS7-Cl)

PolyAPS7-Cl could be synthesised using microwave-assisted polymerisation of the monomer unit, 3-(7-chloroheptyl)pyridine (10) in MeOH and following Scheme 2. The monomer (10) could be prepared from 6-bromo-l-hexanol (2) using tetrahydropyran as a protecting group. The intermediate 7-(3-pyridyl)-heptanol (5) could be then transformed into the monomer, 3-(7-chloroheptyl)pyridine (10), by reaction with thionyl chloride at room temperature.

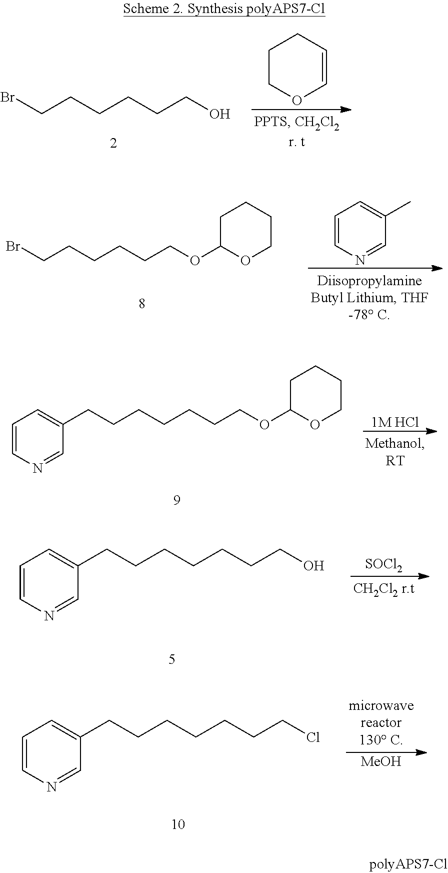

### EXAMPLE 3

### Synthesis of poly- (3- (12-bromododecyl)pyridine) polyAPS12-Br

PolyAPS12-Br was synthesised using microwave-assisted polymerisation of the monomer unit, 3-(12-bromododecyl) pyridine (16) in MeOH and following Scheme 3. The monomer could be prepared from 11-bromo-l-undecanol (11), by protection with a silyl group and coupling with 3-picoline, then de-protection to give the intermediate 12-(3-pyridyl)-1-dodecanol (14), which could be transformed into 3-(12-bromododecyl)pyridine bromide (15) by reacting with hydrobromic acid. The monomer salt (15) was neutralised just prior to polymerisation.

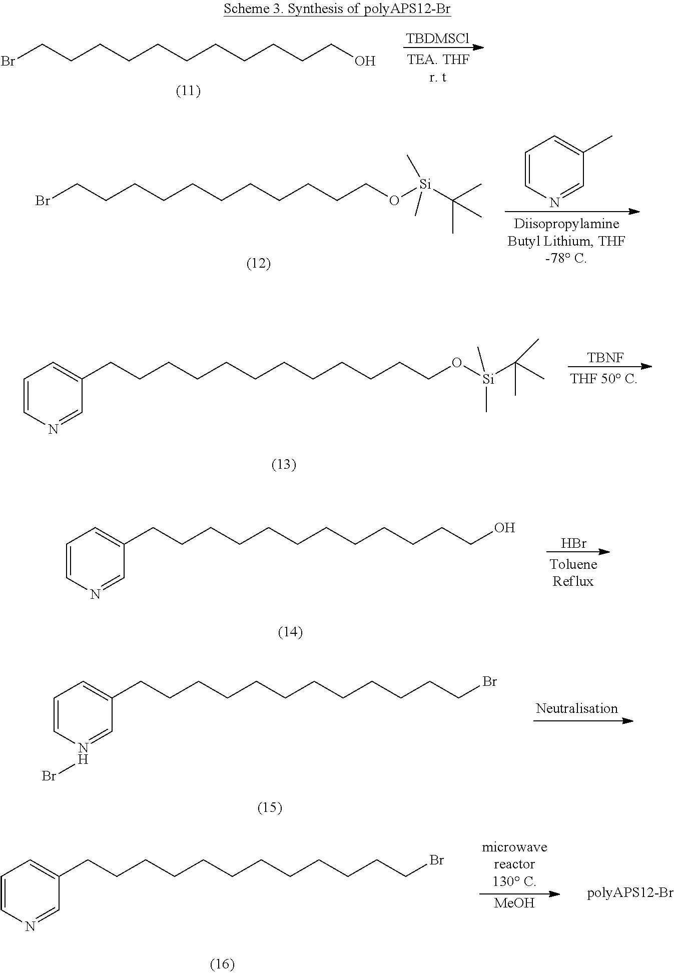

### EXAMPLE 4

### Synthesis of poly- (3- (12-chlorododecyl)pyridine) polyAPS12-Cl

PolyAPS12-Cl was synthesised using microwave-assisted polymerisation of the monomer unit, 3-(12-chlorododecyl)pyridine (17) and following Scheme 4.

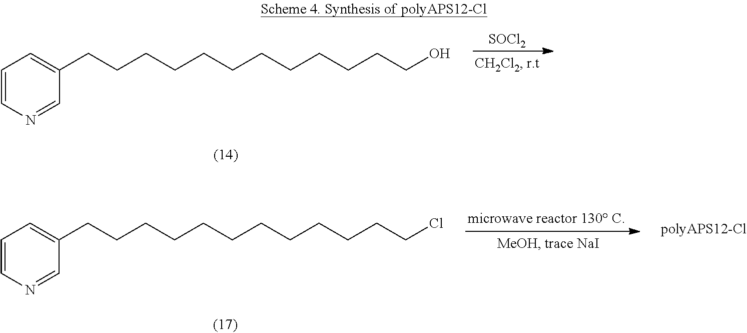

### EXAMPLE 5

### Synthesis of poly- (3-(8-bromooctyl)pyridine) polyAPS8-Br

PolyAPS8-Br was synthesised using microwave-assisted polymerisation of the monomer unit, 3-(8-bromooctyl)pyridine (19) and following Scheme 5.

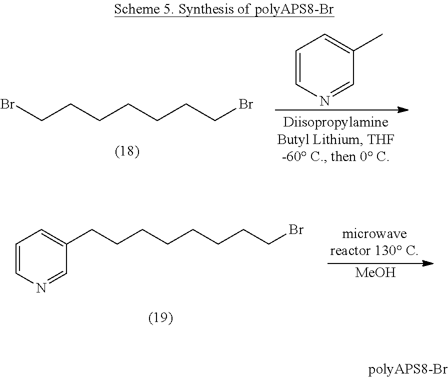

### EXAMPLE 6

### Synthesis of poly- (3-(3-chloropropyl)pyridine) polyAPS3-Cl

PolyAPS3-Cl was synthesised using microwave-assisted polymerisation of the monomer unit, 3-(3-chloropropyl)pyridine (21) and following Scheme 6.

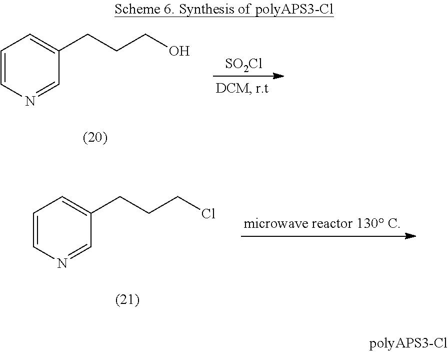

### Determination of Molecular Weights and Structure

For the natural poly-APS, molecular weight determination is carried out using MALDI-TOF mass spectrometry which gives reliable and consistent results. However, for the synthetic poly-APS this method gave variable results, and hence an alternative method was found to obtain molecular weight data on these synthetic poly-APS. The method applied was high resolution electrospray mass spectrometry with an Orbitrap mass analyser. The data for the synthetic poly-APS described above is given below and indicates the monodispersity and high degree of polymerisation and molecular weight.

In FIGS. 2, 3 and 4 the data for polyAPS8-Br and two different polymerisation attempts of polyAPS12-Br of the present invention are presented. All are high molecular weight with 63, 51 and 60 monomer units respectively and highly monodisperse with significantly more than half of the polymer made up of the high molecular weight (11-15 kDa) polymer.

Evidence for the cyclic nature of example polymers of the present invention is indicated by mass spectrometric analysis and ¹H NMR spectra. For example, non-detection of signals for terminal methyl or pyridine groups in ¹H NMR spectra of example polymers can suggests a cyclic nature to these compounds. Previous studies of niphatoxins have illustrated that the ¹H NMR signals of terminal pyridine groups should be easily differentiated from those of the pyridinium rings.

### Experimental

−H NMR or ¹³C-NMR spectra were recorded in CDCl₃ or CD₃OD (as specified) on a Bruker AC 250 NMR spectrometer at 250 MHz. Chemical shifts were reported as 6 values relative to an internal standard, tetramethylsilane (0 ppm). Coupling constants are given in Hz. The microwave-assisted polymerisations were performed on Biotage Initiator Microwave Synthesizer. All starting materials were purchased from Aldrich.

### EXAMPLE 7

### 6-Bromo-1-hexanol (2)

1,6-hexandiol (1) (35.6 g, 0.3 mol), hydrobromic acid (aq. 48%, 51 g, 0.3 mol) and toluene (100 mL) were mixed in a round-bottom flask equipped with a Dean-Stark trap. The mixture was heated under reflux with stirring over two days. After that, the mixture was concentrated and subjected to flash chromatography using petroleum ether 100% followed by petroleum ether : dichloromethane (1:1 v/v) and finally dichloromethane 100% as a mobile phase to give 6-bromo-1-hexanol (2) as a pale yellow liquid (32.4 g, 60%).

¹H-NMR (CDCl₃) : 3.66 (t, 2H, CH₂OH, J=6.4 Hz), 3.40 (t, 2H, BrCH₂, J=6.7 Hz), 2.5 (brs, 1H, OH), 1.86 (m, 2H, CH₂), 1.30-1.70 (m, 6H, CH2).

¹³C-NMR CDCl3): 62.9, 33.9, 32.7, 32.4, 28.0 and 25.0.

### EXAMPLE 8

### 6-Bromohexyl tert-butyldimethylsilyl ether (3)

A mixture of 6-bromo-1-hexanol (2) (15 g, 83 mmol), tert-butyldimethylsilyl chloride (10.8 g, 72 mmol), triethylamine (15.2 g) and trace of 4-dimethylaminopyridine in dichloromethane (150 mL) was stirred overnight to form a white precipitate. The reaction mixture was then washed with water, and the organic layer was concentrated and subjected to flash chromatography on silica gel using dichloromethane 100% as a mobile phase to give 6-bromohexyl tert-butyldimethylsilyl ether (3) (17.0 g, 80%) as a pale yellow oil.

¹H-NMR (CDCl₃) : 3.61 (t, 2H, CH₂OSi, J=6.3 Hz), 3.40 (t, 2H, BrCH₂, J=6.8 Hz), 1.85 (m, 2H, CH₂), 1.30-1.70 (m, 6H, CH₂), 0.89 (s, 9H, SiC(CH₃)₃), 0.05 (s, 6H, Si(CH₃)₂).

¹³C-NMR (CDCl₃): 62.9, 33.9, 32.7, 32.4, 28.0, 25.0, 18.4 and −5.3.

### EXAMPLE 9

### 7-(3-Pyridyl)heptyl tert-butyldimethylsilyl ether (4)

A mixture of 3-picoline (10.6 g, 0.11 mol), diisopropylamine (10.0 g, 0.10 mol) and 6-bromohexyl tert-butyldimethylsilyl ether (3) (22.0 g, 0.075 mol) in tetrahydrofuran (200 mL) was cooled down to −60° C. with stirring. Butyllithium in hexane (2.5 M, 70 mL) was added to the mixture drop wise over 1 hour. The mixture was then slowly warmed to 0° C. and kept at 0° C. overnight. The reaction mixture was then quenched with saturated ammonium chloride, washed with water followed by brine, dried over magnesium sulphate and concentrated. The crude product was subjected to flash chromatography on silica gel using dichloromethane 100% as a mobile phase to give 4 as a brown oil (14.1 g, 62%).

¹H-NMR (CDCl₃) : 8.41 (s, 2H, ArH), 7.54 (m, 1H, ArH), 7.20 (m, 1H, ArH), 3.57 (t, 2H, SiOCH₂, J=6.4 Hz), 2.58 (t, 2H, ArCH₂, J=7.3 Hz), 1.1-2.0 (m, 10H, CH₂), 0.87 (s, 9H, C(CH₃)), 0.03 (s, 6H, SiCH₃).

¹³C-NMR (CDCl₃): 150.0, 147.2, 137.8, 135.8, 123.2, 63.2, 33.0, 32.8, 31.1, 29.2, 29.1, 26.0, 25.8, 18.4 and −5.3.

### EXAMPLE 10

### 7-(3-Pyridyl)heptanol (5)

Compound 4 (13.1 g) was dissolved in tetrahydrofuran (120 mL) and reacted with tetrabutylammonium fluoride (10.8 g) at room temperature for 2 hours. The reaction mixture was washed with water and brine and the water layer was extracted with ethyl acetate. The organic layer was concentrated and subjected to flash column chromatography on silica gel using dichloromethane 100% followed by 5% methanol in dichloromethane as eluent to give 7-(3-pyridyl)heptanol (5) as a light brown oil (6.6 g, 80%).

¹H-NMR (CDCl₃): 8.41 (s, 2H, ArH), 7.54 (m, 1H, ArH), 7.20 (m, 1H, ArH), 3.62 (t, 2H, CH₂OH, J=6.4 Hz), 2.58 (t, 2H, ArCH₂, J=7.3 Hz), 1.1-2.0 (m, 10H, CH₂).

¹³C-NMR (CDCl₃): 150.0, 147.2, 137.9, 135.8, 123.2, 62.9, 33.0, 32.8, 31.0, 29.2, 29.1, 26.0 and 25.7.

### EXAMPLE 11

### 3-(7-bromoheptyl)pyridine bromide (6)

A mixture of 7-(3-pyridyl)heptanol (5) (3.7 g, 20 mmol) and hydrobromic acid (4.1 g) in toluene (20 g) was heated under reflux overnight. The resulting mixture was then subjected to flash column chromatography on silica gel using dichloromethane 100% followed by 5% methanol in dichloromethane as eluent to give 3-(7-bromoheptyl)pyridine bromide (6) as a brown oil.

¹H-NMR (CDCl₃) : 8.68 (brs, 2H, ArH), 8.26 (m, 1H, ArH), 7.91 (m, 1H, ArH), 3.39 (t, 2H, BrCH₂, J=6.4 Hz), 2.84 (t, 2H, ArCH₂, J=7.3 Hz), 1.1-2.0 (m, 10H, CH₂).

¹³C-NMR (CDCl₃): 150.0, 147.3, 137.9, 135.8, 123.2, 34.0, 33.0, 32.7, 31.0, 28.9, 28.6 and 28.0.

### EXAMPLE 12

### 3-(7-Bromoheptyl)pyridine (7)

3-(7-Bromoheptyl)pyridine (7) can be obtained by neutralisation of 6 just prior to the polymerisation.

¹H-NMR (CDCl₃) : 8.41 (s, 2H, ArH), 7.54 (m, 1H, ArH), 7.20 (m, 1H, ArH), 3.39 (t, 2H, BrCH₂, J=6.4 Hz), 2.58 (t, 2H, ArCH₂, J=7.3 Hz), 1.1-2.0 (m, 10H, CH₂).

### EXAMPLE 13

### Poly-(3-(7-bromoheptyl)pyridine); polyAPS7-Br

The monomer, 3-(7-bromoheptyl)pyridine (7) (0.42 g) was dissolved in methanol (2.0 g) and subjected to microwave-assisted polymerisation (130° C., 8 bar, 30 w) for 12 hours (monitored by ¹H-NMR, CDCl₃/CD₃OD). The resulting mixture was concentrated and extracted with a mixture of petroleum ether and dichloromethane (1:1 v/v) to remove unreacted monomer and some low molecular weight oligomers. Final purification was carried out by size exclusion on sephadex LH-20 to give polyAPS7-Br as a brown oil (0.3 g).

¹H-NMR (CDCl₃) : 9.18 (1H, ArH), 8.97 (1H, ArH), 8.20 (1H, ArH), 7.88 (1H, ArH), 4.65 (2H, CH₂Cl), 2.78 (2H, pyCH₂), 1.0-1.70 (10H, CH₂).

### EXAMPLE 14

### 3-(7-chloroheptyl)pyridine (10)

Thionyl chloride (4.2 g, 36 mmol, 1.2 equiv.) was added drop wise to a solution of 7-(3-pyridyl)heptanol (5) (5.8 g, 30 mmol) in dichloromethane (25 mL) at room temperature over half an hour. After that, the mixture was stirred for another two hours. The solution was neutralised with 2M potassium carbonate and the organic layer was dried with magnesium sulphate, and concentrated. The residue was subjected to flash chromatography on silica gel using a mixture of petroleum ether and ethyl acetate (1:1 v/v) followed by ethyl acetate 100% as eluent to give 3-(7-chloroheptyl)pyridine (10) (4.0 g, 63%).

¹H-NMR (CDCl₃): 8.41 (s, 2H, ArH), 7.46 (m, 1H, ArH), 7.20 (m, 1H, ArH), 3.51 (t, 2H, ClCH₂, J=6.4 Hz), 2.58 (t, 2H, ArCH₂, J=7.3 Hz), 1.2-1.9 (m, 10H, CH₂).

¹³C-NMR (CDCl₃): 150.0, 147.3, 137.8, 135.8, 123.2, 45.1, 33.0, 32.6, 31.0, 29.0, 28.7 and 26.8.

### EXAMPLE 15

### Poly-(3-(7-chloroheptyl)pyridine), polyAPS7-Cl

The monomer, 3-(7-chloroheptyl)pyridine (10) (4.0 g) was dissolved in methanol (8.0 g) and subjected to microwave-assisted polymerisation (130° C., 8 bar, 30 w) for 60 hours (monitored by ¹H-NMR, CDCl₃/CD₃OD). The resulting mixture was concentrated and extracted with a mixture of petroleum ether and dichloromethane (1:1 v/v) to remove unreacted monomer and some oligomers. Final purification was carried out by size exclusion on sephadex LH-20 to give polyAPS7-Cl as brown oil (3.5 g).

¹H-NMR (CDCl₃) : 9.18 (1H, ArH), 8.97 (1H, ArH), 8.20 (1H, ArH), 7.88 (1H, ArH), 4.65 (2H, CH₂Cl), 2.78 (2H, pyCH₂), 1.0-1.70 (10H, CH₂).

### EXAMPLE 16

### 11-bromo-1-undecyl tert-butyldimethylsilyl ether (12)

A mixture of 11-bromo-l-undecanol (11) (10.6 g, 42 mmol), tert-butyldimethylsilyl chloride (6.8 g, 45 mmol), triethylamine (5.0 g) and trace of 4-dimethylaminopyridine in dichloromethane (100 mL) was stirred overnight to form a white precipitate. The reaction mixture was then concentrated and subjected to flash chromatography on silica gel using dichloromethane 100% as a mobile phase to give 11-bromo-1-undecyl tert-butyldimethylsilyl ether (12) (14.6 g, 95%) as pale yellow oil.

¹H-NMR (CDCl₃) : 3.58 (t, 2H, CH₂OSi, J=6.3 Hz), 3.39 (t, 2H, BrCH₂, J=6.8 Hz), 1.85 (m, 2H, CH₂), 1.30-1.70 (m, 16H, CH₂), 0.89 (s, 9H, SiC(CH₃)₃), 0.05 (s, 6H, Si(CH₃)₂).

¹³C-NMR (CDCl₃): 63.4, 34.1, 32.9, 32.7, 29.6, 29.5, 29.4, 28.9, 28.8, 28.2, 26.9, 26.0, 25.8, 18.4 and −5.2.

### EXAMPLE 17

### 12-(3-Pyridyl)dodecyl tert-butyldimethylsilyl ether (13)

A mixture of 3-picoline (5.0 g, 54 mmol), diisopropylamine (5.9 g, 57 mmol) and 11-bromo-l-undecyl tert-butyldimethylsilyl ether (14.6 g, 40 mmol) in tetrahydrofuran (200 mL) was cooled down to −60° C. with stirring. Butyllithium in hexane (2.5 M, 40 mL) was then added drop wise over 1 hour. The mixture was then slowly warmed to 0° C. and kept at 0° C. overnight. The reaction mixture was quenched with saturated ammonium chloride, washed with water followed by brine, dried over magnesium sulphate and concentrated. The crude product was subjected to flash chromatography on silica gel using dichloromethane 100% as a mobile phase to give 13 as brown oil (12.0 g, 80%).

¹H-NMR (CDCl₃) : 8.41 (brs, 2H, ArH), 7.47 (d, 1H, ArH, J=7.4 Hz), 7.20 (m, 1H, ArH), 3.58 (t, 2H, SiOCH₂, J=6.4 Hz), 2.58 (t, 2H, ArCH₂, J=7.3 Hz), 1.1-2.0 (m, 20H, CH₂), 0.87 (s, 9H, SiC(CH₃), 0.03 (s, 6H, SiCH₃).

¹³C-NMR (CDCl₃): 150.0, 147.1, 138.0, 135.9, 123.2, 63.4, 33.0, 32.9, 31.2, 31.1, 29.6, 29.5, 29.4, 29.2, 29.1, 26.3, 26.0, 25.8, 18.4 and −5.2.

### EXAMPLE 18

### 12-(3-Pyridyl)dodecanol (14)

A solution of 13 (12.0 g) in tetrahydrofuran (120 mL) was treated with tetrabutylammonium fluoride (6.2 g) at room temperature for 2 hours. The reaction mixture was washed with water and brine and the aqueous mixture was extracted with ethyl acetate. The organic layer was concentrated and subjected to flash chromatography on silica gel using dichloromethane 100% followed by 5% methanol in dichloromethane as eluent to give 14 as light brown oil (7.5 g, 90%).

¹H-NMR (CDCl₃) : 8.42 (s, 2H, ArH), 7.48 (d, 1H, ArH, J=7.8 Hz), 7.20 (m, 1H, ArH), 3.63 (t, 2H, CH₂OH, J=6.4 Hz), 2.59 (t, 2H, ArCH₂, J=7.3 Hz), 1.1-1.8 (m, 20H, CH₂).

¹³C-NMR (CDCl₃): 150.0, 147.1, 138.0, 136.0, 123.3, 62.9, 33.0, 32.8, 31.1, 29.6, 29.5, 29.4, 29.1 and 25.8.

### EXAMPLE 19

### 3-(12-bromododecyl)pyridine bromide (15)

A mixture of 12-(3-pyridyl)-1-undecanol (14) (7.5 g, 28 mmol) and hydrobromic acid (18.5 g) in toluene (15 g) was heated under reflux overnight. The resulting mixture was subjected to flash column chromatography on silica gel using dichloromethane 100% followed by 5% methanol in dichloromethane as an eluent and re-crystallised from acetone to give 15 as an off-white solid.

¹H-NMR (CDCl₃) : 8.68 (brs, 2H, ArH), 8.26 (m, 1H, ArH), 7.91 (m, 1H, ArH), 3.39 (t, 2H, BrCH₂, J=6.4 Hz), 2.84 (t, 2H, ArCH₂, J=7.3 Hz), 1.1-2.0 (m, 20H, CH₂). ¹³C-NMR (CDCl₃): 146.4, 143.4, 140.0, 138.3, 126.9, 34.2, 32.9, 32.8, 30.4, 29.5, 29.4, 29.2, 29.0, 28.9, 28.7 and 28.2.

### EXAMPLE 20

### 3-(12-bromoheptyl)pyridine (16)

3-(12-bromoheptyl)pyridine (16) can be obtained as a yellowish oil by neutralisation of 15 just prior to the polymerisation.

¹H-NMR (CDCl₃): 8.42 (s, 2H, ArH), 7.47 (m, 1H, ArH), 7.18 (m, 1H, ArH), 3.39 (t, 2H, BrCH₂, J=6.7 Hz), 2.58 (t, 2H, ArCH₂, J=7.3 Hz), 1.83 (m, 2H, BrCH₂CH₂), 1.1-1.7 (m, 18H, CH₂).

¹³C-NMR (CDCl₃): 150.0, 147.1, 138.0, 136.0, 123.3, 34.2, 33.0, 32.8, 31.2, 29.6, 29.5, 29.4, 29.2, 28.9, 28.8 and 28.2.

### EXAMPLE 21

### Poly- (3-(12-bromododecyl)pyridine); polyAPS12-Br

3-(12-bromoheptyl)pyridine (16) (0.5 g) was dissolved into dry acetonitrile (6 mL) and heated under reflux for 2 days. At this stage a small amount of sodium iodide (0.1 g) was added and the solution was heated under reflux for 2 more days. Another amount of sodium iodide (0.5 g, excess) was added and the reflux was continued for another 3 days. After the polymerisation, more acetonitrile (5 mL) was added to the solution and filtered off. The acetonitrile solution was concentrated to give a yellowish waxy solid. Micro-wave assisted polymerisation (130° C., 8 bar, 40 w) of that solid in methanol over 30 minutes gave the viscous polymer polyAPS12-Br (12.5 kDa). Micro-wave assisted polymerisation (130° C., 8 bar, 40 w) of polyAPS12-Br (12.5 kDa) in methanol over two days give the more viscous polymer polyAPS12-Br (15 kDa).

¹H-NMR (CD₃OD) : 9.07 (brs, 1H, ArH), 8.88 (m, 1H, ArH), 8.48 (m, 1H, ArH), 8.03 (m, 1H, ArH), 4.68 (m, 2H, ArNCH₂), 2.93 (m, 2H, ArCH₂), 2.04 (m, 2H, ArNCH₂CH₂), 1.74 (m, 2H, ArCH₂CH₂), 1.0-1.5 (m, 16H, CH₂).

### EXAMPLE 22

### 3-(12-chlorododecyl)pyridine (17)

Thionyl chloride (2.3 g, 20 mmol, 2.0 equiv.) was added drop wise to a solution of 12-(3-pyridyl)-1-dodecanol (14) (2.63 g, 10 mmol) in dichloromethane (25 mL), at room temperature over 30 min. The mixture was then stirred for another two hours. After that, the solution was neutralised with 2 M potassium carbonate aqueous solution. The organic layer was dried with magnesium sulphate, and concentrated. The residue was subjected to flash chromatography on silica gel using a mixture of petroleum ether and ethyl acetate (1:1 v/v) followed by ethyl acetate 100% as a mobile phase to give 3-(12-chlorododecyl)pyridine (17) (1.97 g, 70%).

¹H-NMR (CDCl₃): 8.41 (s, 2H, ArH), 7.46 (m, 1H, ArH), 7.20 (m, 1H, ArH), 3.51 (t, 2H, ClCH₂, J=6.4 Hz), 2.58 (t, 2H, ArCH₂, J=7.3 Hz), 1.2-1.9 (m, 20H, CH₂).

¹³C-NMR (CDCl₃): 150.0, 147.3, 137.8, 135.8, 123.2, 45.1, 33.0, 32.8, 31.2, 29.6, 29.5, 29.4, 29.2, 28.9, 28.8 and 28.2.

### EXAMPLE 23

### Poly- (3-(12-chloroheptyl)pyridine); polyAPS12-Cl

3-(12-chloroheptyl)pyridine (17) (0.5 g) in methanol (4.0 g) was subjected to microwave-assisted polymerisation (130° C., 8 bar, 30 w) for 60 hours (monitored by ¹H-NMR, CDCl₃/CD₃OD). The resulting mixture was concentrated and extracted with a mixture of petroleum ether and dichloromethane (1:1 v/v) to remove un-reacted monomer and some oligomers. Final purification was carried out by size exclusion on sephadex LH-20 column to give polyAPS12-Cl as brown oil (0.40 g).

¹H-NMR (CDCl₃) : 9.18 (1H, ArH), 8.97 (1H, ArH), 8.20 (1H, ArH), 7.88 (1H, ArH), 4.65 (2H, CH₂Cl), 2.78 (2H, pyCH₂), 1.0-1.70 (20H, CH₂).

### EXAMPLE 24

### 3-(8-bromooctyl)pyridine hydrochloride salt

A mixture of 3-picoline (4.4 g, 47 mmol), diisopropylamine (4.7 g, 47 mmol) and 1,7-dibromoheptane (18) (11.1 g) in tetrahydrofuran (200 mL) was cooled down to -60° C. with stirring. Butyllithium in hexane (2.5 M, 30 mL) was added drop wise over 1 hour. After that, the mixture was slowly warmed to 0° C. and kept at 0° C. overnight. The reaction mixture was concentrated and dissolved in methanol. Diluted hydrochloric acid was added to the mixture to give 3-(8-bromooctyl)pyridine hydrochloride salt. The salt was purified by flash chromatography on silica gel using dichloromethane 100% followed by 5% methanol in dichloromethane as eluent to give a brown oil (5.0 g, 35%).

¹H-NMR (CDCl₃) : 8.68 (m, 2H, ArH), 8.26 (m, 1H, ArH), 7.91 (m, 1H, ArH), 3.38 (t, 2H, BrCH₂, J=6.4 Hz), 2.84 (t, 2H, ArCH₂, J=7.3 Hz), 1.1-2.0 (m, 12H, CH₂).

### EXAMPLE 25

### 3-(8-bromooctyl)pyridine (19)

¹H-NMR (CDCl₃) : 8.43 (s, 2H, ArH), 7.48 (d, 1H, ArH, J=7.8 Hz), 7.20 (dd, 1H, ArH), 3.39 (t, 2H, BrCH₂, J=6.9 Hz), 2.59 (t, 2H, ArCH₂, J=7.3 Hz), 1.1-2.0 (m, 12H, CH₂).

¹³C-NMR (CDCl₃): 150.0, 147.3, 137.9, 135.8, 123.3, 34.1, 33.0, 32.8, 31.1, 29.2, 28.7, 28.1

### EXAMPLE 26

### Poly-(3-(8-bromooctyl)pyridine); polyAPS8-Br

A solution of 3-(8-bromooctyl)pyridine (19) (4.0 g) in methanol (8.0 g) was subjected to microwave-assisted polymerisation (130° C., 8 bar, 30 w) for 60 hours (monitored by ¹H-NMR, CDCl₃/CD₃OD). The resulting mixture was concentrated and extracted with a mixture of petroleum ether and dichloromethane (1:1 v/v) to remove un-reacted monomer and some oligomers. Final purification was carried out by size exclusion on sephadex LH-20 to give polyAPS8-Br as brown oil (3.5 g).

¹H-NMR (CDCl₃) : 9.18 (1H, ArH), 8.97 (1H, ArH), 8.20 (1H, ArH), 7.88 (1H, ArH), 4.65 (2H, CH₂Cl), 2.78 (2H, pyCH₂), 1.0-1.70 (12H, CH₂).

### EXAMPLE 27

### 3-(3-chloropropyl)pyridine (21)

Thionyl chloride (4.2 g, 36 mmol, 1.2 equiv.) was added drop wise to a solution of 3-(3-hydroxypropyl)-pyridine (20) (4.1 g, 30 mmol) in dichloromethane (25 mL) at room temperature over 30 min. After that, the mixture was stirred for another two hours and the solution was neutralised with 2M potassium carbonate aqueous solution. The organic layer was dried with magnesium sulphate, and concentrated. The residue was subjected to flash chromatography on silica gel using a mixture of petroleum ether and ethyl acetate (1:1 v/v) to give 3-(3-chloropropyl)pyridine (21) (4.0 g, 85%).

¹H-NMR (CDCl₃) : 8.47 (s, 2H, ArH), 7.51 (d, 1H, J=7.8 Hz, ArH), 7.22 (dd, 1H, J=7.8, 4.5 Hz, ArH), 3.52 (t, J=7.2 Hz, CH₂Cl), 2.79 (t, J=7.3 Hz, pyCH₂), 2.08 (2H, m, pyCH₂CH₂CH₂);

¹³C-NMR (CDCl₃): 150.0, 147.8, 136.0, 123.5, 43.9, 33.6, 29.9

### EXAMPLEe 28

### Poly-(3-(3-chloropropyl)pyridine); polyAPS-Cl

A solution of 3-(3-chloropropyl)pyridine (4.0 g) in methanol (8.0 g) with trace of sodium iodide was subjected to microwave-assisted polymerisation (130° C., 8 bar, 30 w) for 60 hours (monitored by 1H-NMR, CDCl₃/CD₃OD). The resulting mixture was concentrated and extracted with a mixture of petroleum ether and dichloromethane (1:1 v/v) to remove un-reacted monomer and some oligomers. Final purification was carried out by size exclusion on sephadex LH-20 column to give polyAPS3-Cl as brown oil (3.5 g).

¹H-NMR (CDCl₃) : 9.41 (1H, ArH), 9.07 (1H, ArH), 8.46 (1H, ArH), 7.85 (1H, ArH), 4.72 (2H, CH₂Cl) 3.00 (2H, pyCH₂), 2.46 (2H, pyCH₂CH₂CH₂).

¹³C-NMR (CDCl₃): 146.0.0, 144.7, 142.2, 128.0, 60.5, 31.8 and 28.9.

### Biology Section

In the following Examples the synthetic poly-APS were screened for a variety of biological activities which included antibacterial and haemolytic actions, anti-acetylcholinesterase activity and toxicity. These biological activities have previously been reported for the natural toxin. Additionally, undifferentiated mouse embryonic stem cells were used to evaluate the ability of the two compounds polyAPS12-Br (APS12, 12.5 kDa) and polyAPS12-Br (APS12-2, 15 kDa) to make ion permeable pores in cell membranes. The actions of the synthetic compounds have been compared with those of natural poly-APS. A main goal of the project was to develop new synthetic pore-forming compounds that could be used as novel transfection reagents for the delivery of DNA into cells. Embryonic stem cells were selected as a model for this project because as part of the study we were interested to see whether exposure to poly-APS would have long-term effects on cells. The embryonic cells can be stimulated with retinoic acid to undergo the complex processes of differentiation into immature GABAergic neurones. Differentiation can be monitored over several weeks as cell phenotypes change and potential long-term influences of exposure to poly-APS determined. Our previous work showed that small polymers of alkylpyridinium salts did not form pores in membranes and had distinct actions compared to poly-APS. Thus, our aim was to compare the actions of natural poly-APS with those of the structurally related large synthetic polymers polyAPS12-Br (APS-12, 12.5 kDa) and polyAPS12-Br (APS12-2, 15 kDa).

### EXAMPLE 29

Antibacterial Activity of the Synthetic poly-APS Analogues

The antibacterial activity of each test compound against E. coli and S. aureus was evaluated and each MIC was determined.

Consistent with previous work with the natural poly-APS, the synthetic analogues showed antibacterial activity against E. coli and S. aureus (Table 1).

TABLE 1
Minimal inhibitory concentrations (mg/mL):

| Compound with counter ion | E. coli | S. aureus |
|---|---|---|
| PolyAPS7-Cl | 0.5 | 0.07 |
| PolyAPS12-Br (12.5 KDa) | 5 | 0.3 |
| PolyAPS12-Br (15 KDa) | 0.5 | 0.1 |
| PolyAPS12-Cl | 0.3 | 0.03 |
| PolyAPS8-Br | 0.3 | 0.05 |
| PolyAPS3-Cl | 10 | 1 |

### EXAMPLE 30

### Haemolytic Activity

Haemolysis was measured by means of a turbidimetric method. Bovine red blood cells were washed in erythrocyte buffer three times. Erythrocytes were centrifuged each time and after final centrifugation cells were re-suspended in erythrocyte buffer to give an apparent absorption of 0.500 units (+/−0.10). Upon addition of 1 μg of each compound, haemolysis was monitored spectrophotometrically until turbidity disappeared and clear haemolysed solution was obtained. The rate of haemolysis was expressed as 1/t₅₀ (s⁻¹).

The haemolytic activity of the synthetic poly-APS was analysed to obtain the time course of haemolysis for each compound. In all cases the compounds produced sigmoidal time/haemolysis relationship which indicated the coloid-osmotic mechanism of cell lysis (Table 2 and FIG. 6). This mechanism is observed when the binding formation of transient pores with defined radius might occur. This is corroborated by the experiments with osmoprotectants where osmoprotectants with larger mol. w. (i.e. Peg 600) showed considerable slow down in the rate of haemolysis.

Table 2 shows comparisons of haemolysis rates produced by 1 μg of each test compound. All compounds tested produced detectable haemolysis rates at this dose except for APS3 which was inactivate at this dose.

TABLE 2

| | Haemolysis 1/t₅₀ | |
|---|---|---|
| Compound with counter ion | (1 μg) | Type (time course) |
| PolyAPS7-Cl | 0.01 s⁻¹ | sigmoidal |
| PolyAPS12-Br (12.5 KDa) | 0.08 s⁻¹ | sigmoidal |
| PolyAPS12-Br (15 KDa) | 0.1 s⁻¹ | sigmoidal |
| PolyAPS12-Cl | 0.013 s⁻¹ | sigmoidal |
| PolyAPS8-Br | 0.02 s⁻¹ | sigmoidal |
| PolyAPS3-Cl | not measurable | almost no activity |

### EXAMPLE 31

Acetylcholine esterase (AChE) Inhibition

The activity of electric eel acetyl cholinesterase (AChE) was measured at three concentrations of substrate (ACh: 0.5, 0.25 and 0.125 mM final concentration, respectively). Controls were made with the same ingredients without the enzyme. Measurements were done according to Ellman's method using a microplate reader instrument. Final volume of each reaction mixture was 200 μl. Results are presented as Dixon plots. Type of inhibition and corresponding Kᵢ were calculated from the obtained plots.

Similar to natural poly-APS the synthetic analogues inhibited AChE activity (Table 3 and FIG. 5). The length of the alkyl chain appears important to inhibitory activity of the synthetic compounds. The longer the chain length of the compound, the higher its inhibitory action on AChE. Molecular weight may also play an important role since it directly affects the type of AChE inhibition. Smaller molecules with shorter alkyl chain act as competitive inhibitors but their inhibitory activity is smaller (larger inhibitory constant, Kᵢ).

TABLE 3
Type of inhibition and Kᵢ for different synthetic
poly-APS analogues

| Compound with counter | | |
|---|---|---|
| ion | Kᵢ | Type of inhibition |
| PolyAPS7-Cl | 25 ng/ml | noncompetitive |
| PolyAPS12-Br (12.5 KDa) | 0.2 ng/mL 3.5 × 10⁻¹⁰ M | noncompetitive |
| PolyAPS12-Br (15 KDa) | 0.5 ng/mL | noncompetitive |
| PolyAPS12-Cl | 12 ng/mL | noncompetitive |
| PolyAPS8-Br | 15 ng/mL | noncompetitive |
| PolyAPS3-Cl | 124 ng/mL | competitive |

### Method—Cell Culture

HEK 293 cells were maintained in culture. Briefly, cells were cultured in EMEM, supplemented with 10% FCS, 2 mM L-glutamine, 50 U/mL penicillin, 50 μg/mL streptomycin and 1% NEA.

The mouse embryonic stem cell line, Abdn2 was derived from C57B1/6JCrl mouse and used in this study. Cells were maintained on mitotically inactivated MEF, in the KOSR-KDMEM medium comprising of Knockout DMEM (Invitrogen) supplemented with 20% Knockout Serum Replacement (Invitrogen), 0.1 mM β-mercaptoethanol (Sigma), purified recombinant mouse LIF (equivalent to 1000 U/mL), 0.1 mM NEA (Sigma), 4 mM GlutaMAX™-I (Invitrogen), 50 U/mL penicillin and 50 μg/mL streptomycin (Invitrogen). The media were renewed every two days. The cultures were passaged at −70% of confluence by trypsinization.

Method—RA-Based Neuronal Differentiation Protocol of ES Cells through EB Formation

MEFs were firstly removed prior to EB formation by sub-culturing on gelatinised (0.1%) plates. Sub-confluent cells were transferred on non-adherent 10-cm Petri dishes at a concentration of 2-4×10³ cells/mL in EB growth medium comprising of DMEM supplemented with 10% FCS, 0.1 mM NEA, mM GlutaMAX™-I, 50 U/mL penicillin and 50 μg/mL streptomycin. The dishes were shaken at a 37° C. incubator at 50 rpm to allow cell aggregation. After 3 days of suspension cultures, uniformly sized EBs suitable for RA induction were formed. EBs were induced for differentiation by shaking on non-adherent 10-cm Petri dishes in fresh EB growth medium with all-trans RA at a final concentration of 10⁻⁶ M. After 3 days, EBs were harvested and plated on culture dishes pre-coated with poly-1-ornithine and fibronectin. The cultures were left overnight in EB formation medium (without RA since) to enhance EB attachment.

Cells were washed three times with NaCl-recording medium before poly-APS was applied at a final concentration of 5 μg/mL for 5, 10 or 20 minutes. Cells were then washed with neuronal induction medium comprising of Neurobasal medium supplemented with B27, bFGF (10 ng/mL) and EGF (10 ng/mL) and containing 10% FCS for 30 sec. Serum was added to inactivate the poly-APS. The cells were then cultured in serum-free neuronal induction medium for three weeks before carrying out the electrophysiological experiments. The medium was renewed every 3 days.

### Method—Crystal Violet Cytotoxicity Assay

HEK 293 and Abdn 2 cells were seeded in 96 well plates at 8000 cells/well and incubated for 24 h at 37° C. in 5% CO₂. The media were replaced with serum-free media with or without one of the test materials and cells were incubated for further 48 h. Each test material was added to eight final concentrations range from 0.5 μg/mL - 1 mg /mL in triplicate. After incubation, adherent cells were fixed in paraformaldehyde and stained in crystal violet dye as previously described; subsequent elution and spectrophotometric analysis quantified the amount of intact cells capable of harbouring dye.

### Method—Transfection Experiments

HEK 293 cells were seeded at 1 x 10⁵ cell/well in 6 well plates in 2 mL EMEM media with FCS for 24 h to reach a plate confluency of 50-60% on the day of transfection. Control transfections were carried out using optimized lipofectamine (Invitrogen Life Technologies) lipid-micelle-mediated transfection protocol as previously reported, which incubates cells with 1-μg cDNA and lipofectamine in the absence of serum for 3 h prior to reintroduction to serum-containing medium. The toxin transfection protocol developed by Tucker and colleagues was used in this study. The protocol involved 5-min serum-free cell incubation with the toxin preparation, followed by addition of 2.5-μg cDNA. After a further 3-h incubation, medium was replaced by standard serum-containing medium. The cells were then cultured for further 48 h. Levels of enhanced green fluorescent protein (EGFP) in the transfected cells were detected and corrected for background fluorescence of the control cells using FACS analysis. The transfection efficiency was calculated based on the percentage of the cells that expressed EGFP (positive cells) in the total number of cells.

### EXAMPLE 32

Cytotoxicity of Natural poly-APS, polyAPS12-Br (12.5 kDa) and polyAPS12-Br (15 kDa)

Experiments were conducted to determine the cytotoxicity of natural poly-APS, polyAPS12-Br (12.5 kDa) and polyAPS12-Br (15 kDa) (1 mg/mL-0.5 μg/mL) in two cell types, HEK 293 cells and undifferentiated mouse embryonic stem cells. The mean EC₅₀ values for natural poly-APS, polyAPS12-Br (12.5 kDa) and polyAPS12-Br (15 kDa) when applied to HEK 293 cells were 3.2 μg/mL, 9.5 μg/mL and 8 μg/mL (n=3) respectively. The mean EC50 values for natural poly-APS, polyAPS12-Br (12.5 kDa) and polyAPS12-Br (15 kDa) when applied to undifferentiated stem cells were 7.5 μg/mL, 22.5 μg/mL and 26 μg/mL (n=3) respectively.

Cytotoxicity experiments indicated that HEK 293 cells are more sensitive to natural poly-APS than to its two synthetic analogues. This allowed the use of both polyAPS12-Br (12.5 kDa) and polyAPS12-Br (15 kDa) at a higher concentration (5 μg/mL) than that of natural poly-APS (1 μg/mL) in the transfection experiments. The FACS analysis indicated that natural poly-APS, polyAPS12-Br (12.5 kDa) and polyAPS12-Br (15 kDa) showed transfection efficiencies of 0.5, 7.63, 6.06% respectively compared to lipofectamine which showed an efficiency of 15.6% when studied in HEK 293 cells. Undifferentiated embryonic stem cells proved more difficult to transfect with cDNA. Natural poly-APS, polyAPS12-Br (12.5 kDa) and polyAPS12-Br (15 kDa) showed transfection efficiencies of 0.29, 0.85, 0.72% respectively compared to lipofectamine which showed an efficiency of 12.9%. The background or control transfection efficiency observed when cDNA was applied alone to undifferentiated embryonic stem cells was 0.18%.

### EXAMPLE 36

Electrophysiological Actions of Natural polyAPS and Synthetic Analogues, polyAPS12-Br (12.5 kDa) and polyAPS12-Br (15 kDa)

The electrophysiological actions of natural polyAPS and synthetic analogues, polyAPS12-Br (12.5 kDa) and polyAPS12-Br (15 kDa), on undifferentiated and differentiated embryonic mouse stem cells were characterized at room temperature (18-20° C.) using the whole cell patch clamp techniques. Resting membrane potentials (RMP), input resistances (IR) and current-voltage relationships under voltage clamp were measured in ES cells and differentiated cells at RAd21. Patch pipettes with resistances of 3-9 MW were made from Pyrex borosilicate glass capillary (Plowden and Thompson Ltd, Dial Glass Works) using a two-stage vertical microelectrode puller (David Kopf Instruments, Tujunca, U.S.A, Model 730). An Axoclamp 2A switching amplifier (Axon Instruments) operated at 18 kHz was used. Patch pipettes were filled with KCl-based solution containing 140 mM KCl, 0.1 mM CaCl₂, 5 mM EGTA, 2 mM MgCl₂, 2 mM ATP and 10 mM HEPES. The pH and osmolarity of the patch pipette solutions were corrected to 7.2 and 310-320 mOsmL⁻¹ with Tris and sucrose respectively. The extracellular bathing solution used contained 130 mM NaCl, 2 mM CaCl₂, 3 mM KCl, 0.6 mM MgCl₂, 1 mM NaHCO₃, 10 mM HEPES and 5 mM glucose. The pH and osmolarity of this extracellular bathing solution were corrected to 7.4 and 320 mOsmL⁻¹ with NaOH and sucrose respectively. Data were captured and stored on digital audiotape using a Biologic digital tape recorder (DTR 1200). Analysis of data was performed off-line using Cambridge Electronic Design voltage clamp analysis software (version 6.0). All voltage-activated K⁺ currents recorded from differentiated stem cells had scaled linear leakage and capacitance currents subtracted to obtain values for the net current. Data are given as mean±standard error of the mean (SEM) and statistical significance was determined using a paired or independent Student's t test as appropriate.

Initially basic electrophysiological experiments demonstrated that natural poly-APS (5 μg/mL) applied for ˜20 s caused a reversible collapse of the resting membrane potential and input resistance of undifferentiated stem cells (FIGS. 7 & 8). Cells were held at −70 mV by constant current injection prior to applying a current step command to evoke an electrotonic potential to standardise the measurement of input resistance. Partial recovery (50% or more) of both the membrane potential and input resistance was observed 10-20 minutes after application of poly-APS (FIG. 9). Due to the low and variable resting membrane potential (−43±2 mV (n=8)) of undifferentiated stem cells subsequent experiments were carried out under voltage clamp. Cells were held at −70 mV and leak currents were evoked by 100 ms to clamp potentials between −140 and +80 mV. All undifferentiated cells studied had linear current-voltage relationships. Poly-APS, polyAPS12-Br (12.5 kDa) and polyAPS12-Br (15 kDa) applied at a concentration of 5 μg/mL evoked inward currents. FIG. 10 shows the values of the mean currents required to hold cells at −70 mV under control conditions and the significantly larger mean current observed in the presence of natural poly-APS and the synthetic compounds. All responses were at least partially reversible and FIG. 11 shows an example current record of a response to natural poly-APS. FIG. 12 shows example records of responses to voltage step commands of +130 mV applied under control conditions during the peak response and after 10 minutes recovery. Natural poly-APS and its two synthetic analogues did not cause the current-voltage relationships to deviate from linearity.

### EXAMPLE 37

Intracellular Ca²⁺ Transients Evoked by poly-APS and its Synthetic Analogues polyAPS12-Br (12.5 kDa) and polyAPS12-Br (15 kDa)

Intracellular Ca²⁺ transients [Ca²⁺], evoked in undifferentiated mouse embryonic stem cells by poly-APS and its synthetic analogues polyAPS12-Br (12.5 kDa) and polyAPS12-Br (15 kDa) were measured as previously reported for studies on halitoxin and poly-APS. Cells were incubated in the dark for 1 hour in NaCl-based extracellular solution containing 0.01 mM fura-2AM (Sigma, 1 mM stock in dimethylformamide). The cells were then washed for 10-20 min with NaCl-based extracellular solution to remove excess fura-2AM, this period allowed time for cytoplasmic de-esterification of the Ca²⁺-sensitive fluorescent dye. The cells were constantly perfused (1-2 mL.min⁻¹) with NaCl-based extracellular solution and viewed under an inverted Olympus BX50W1 microscope with a KAI-1001 S/N 5B7890-4201 Olympus camera attached. The fluorescence ratiometric images from data obtained at excitation wavelengths of 340 and 380 nm were viewed and analysed using OraCal pro, Merlin morphometry temporal mode (Life Sciences resources, version 1.20). One minute applications of 0.05, 0.5 and 5 μg/mL of polyAPS12-Br (12.5 kDa; 4 nM, 40 nM & 400 nM) and polyAPS12-Br (15 kDa; 3.3 nM, 33 nM & 333 nM) in NaCl-based extracellular solution were conducted. [Ca²⁺], levels were allowed to return to baseline prior to an additional application of drug-containing solution. Region of interest (ROI; 1 per cell body) within a given field were pre-selected by means of a transmission image overlay. For data analysis, ratiometric values obtained from Openlab (V. 4.02, Improvision, Coventry, UK) were plotted against time and the peak rise in fluorescence for each ROI was determined, shown as arbitrary fluorescence units in sample traces. All values were converted into (% ΔF/F) with F defined as an average of ten baseline values before drug application. The average % ΔF/F was calculated for each dose and statistically compared with that for other drugs. The percentage of cells which responded towards a given drug application was determined in the following way. The average of the ten baseline fluorescence values was subtracted from the maximum baseline fluorescence to give a value for variance in fluorescence prior to drug application. A drug response was considered to have occurred if the change in fluorescence was greater than four times the value of the baseline variance plus the mean baseline fluorescence. Each experiment was repeated at least two times, using different culture batches. Intracellular Ca²⁺ imaging data are given as means±standard errors of the means (SEM) and statistical significances were determined using a paired or independent Student's t test or ANOVA as appropriate. One-way ANOVA followed by Newmann Keuls post test was used for multiple comparisons. Significance was set at P<0.05=significant; P<0.01=highly significant; P<0.001=very highly significant.

Ca²⁺ imaging experiments showed that mean dose-dependent responses to natural poly-APS, polyAPS12-Br (12.5 kDa) and polyAPS12-Br (15 kDa) (0.05, 0.5 and 5 μg/mL) could be obtained (FIG. 13). The proportion of responding cells also increased as the concentration of compound was increased. However, example cells that failed to respond to all concentrations tested of poly-APS were found. Overall cells were more responsive to the synthetic compounds compared with poly-APS. A greater proportion of cells responded to 0.05 μg/mL and significantly larger Ca²⁺ transients were evoked by the two synthetic compounds at the highest concentration tested. There was a considerable amount of variability in the responses to all three compounds and the records from individual cells in FIGS. 13 and 14 show this.

### CONCLUSIONS

In these Examples we used a number of assays of bioactivity and preparations to evaluate the actions of natural natural poly-APS, and synthetic analogues (including polyAPS12-Br (12.5 kDa) and polyAPSq2-Br (15 kDa)). Natural poly-APS induced reversible pore formation, collapsed membrane potential, reduced input resistance, increased Ca²⁺ permeability and can be used as tranfection reagent. The two synthetic compounds similarly evoked reversible inward currents, increased Ca²⁺ permeability and could be used in transfection studies. Cytotoxicity that may also be indicative of pore formation was observed and the synthetic compounds had similar properties to natural poly-APS. The mechanisms of action to produce inhibition of AChE is distinct from pore formation but importantly activity was seen with both natural and synthetic compounds.

1. A method of producing a di-substituted pyridinium polymer, the method comprising the steps of:
obtaining a pyridine monomer selected from 2, 3, and 4-substituted pyridine monomers of the formula NC₅ R₄—R′—X, wherein R is selected from hydrogen, hydroxyl, and substituted and unsubstituted alkyl, alkoxy, aryl, alkaryl, aralkyl, and alkenyl groups, R′ is a linking group, and X is a leaving group;
and polymerising the 2, 3, or 4-substituted pyridine monomer by microwave-assisted polymerisation.

2. The method of claim 1, wherein the pyridine monomer is a 3-substituted pyridine monomer.

3. The method of claim 1, wherein R′ is selected from an alkylene group, an alkenyl-containing group, an alkynyl-containing group, and a cyclopropanyl-containing group.

4. The method of claim 3, wherein R′ is selected from a group —(CH₂)ₘ—, wherein m is an integer from 2 to 15, a group having from 2 to 15 carbon atoms containing one or more alkenyl groups, a group having from 2 to 15 carbon atoms containing one or more alkynyl groups, and cis- or trans- —(CH₂)ₚ-cyclopropanyl-(CH₂)_{q}— wherein p and q are the same or different and are integers from 1 to 6.

5. The method of claim 4, wherein R′ is selected from methylene, ethylene, propylene, butylene, pentylene, hexyl, heptylene, octylene, nonylene, decylene, undecylene and dodecylene.

6. The method of claim 1, wherein X is selected from a halide, triflate, mesylate and tosylate group.

7. The method of claim 6, wherein X is selected from bromide, chloride and iodide.

8. The method of claim 1, wherein the pyridine monomer is selected from 3-(3-chloropropyl)pyridine, 3-(7-bromoheptyl)pyridine, 3-(7-chloroheptyl)pyridine, 3-(8-bromooctyl)pyridine, 3-(12-bromododecyl)pyridine and 3-(12-chlorododecyl)pyridine.

9. The method of claim 1, which comprises at least one step selected from:
the pyridine monomer is dissolved in methanol prior to polymerisation;
the microwave-assisted polymerisation step is carried out at a temperature between 100oC and 200oC;
the microwave-assisted polymerisation step is carried out from between 20 minutes and 80 hours.
the microwave-assisted polymerisation step is carried out at a pressure of between 7 and 9 bar; and
the microwave-assisted polymerisation step is performed on a Biotage Initiator Microwave Synthesizer.

10. The method of claim 9, which comprises at least one step selected from:
the microwave-assisted polymerisation step is carried out at a temperature of 130° C.;
the microwave-assisted polymerisation step is carried out at a pressure of 8 bar; and
the microwave-assisted polymerisation step is performed on the Biotage Initiator Microwave Synthesizer at a power of 30 to 40 Watts.

11. The method of claim 1 wherein the di-substituted pyridinium polymer produced is selected from cyclic and linear polymers.

12. A di-substituted pyridinium polymer obtained from the method of claim 1.

13-33. (canceled)

34. A di-substituted pyridinium polymer composition comprising polymer chains of the formula
NC₅R₄—R′—[X⁻N C₅R₄—R′]—NC₅R₄—R′X or —[X⁻N′C₅R₄—R′]ₙ—
wherein R is selected from hydrogen, hydroxyl, and substituted or unsubstituted alkyl, alkoxy, aryl, alkaryl, aralkyl, and alkenyl groups,
R^{′} is a linking group,
X is a counter ion,
n is the degree of polymerisation and is between 40 and 70,
wherein at least 50% of the di-substituted pyridinium polymer chains in the composition have the same degree of polymerisation.

35. The di-substituted pyridinium polymer composition of claim 34 wherein at least 55% of the di-substituted pyridinium polymer chains in the composition have the same degree of polymerisation.

36. The di-substituted pyridinium polymer composition of claim 34 wherein n is 50 to 70.

37. The di-substituted pyridinium polymer composition of claim 34, wherein the di-substituted-pyridinium polymer comprises pyridinium rings substituted by R′ at the 3 position.

38. The di-substituted pyridinium polymer composition of claim 34, wherein R′ is selected from an alkylene group, an alkenyl-containing group, an alkynyl-containing group, and a cyclopropanyl-containing group.

39. The di-substituted pyridinium polymer composition of claim 38, wherein R′ is selected from a group —(CH₂)ₘ—, wherein m is an integer from 2 to 15, a group having from 2 to 15 carbon atoms containing one or more alkenyl groups, a group having from 2 to 15 carbon atoms containing one or more alkynyl groups, and cis- or trans- —(CH₂)ₚ-cyclopropanyl-(CH₂)_{q}— wherein p and q are the same or different and are integers from 1 to 6.

40. The di-substituted pyridinium polymer composition of claim 39, wherein R′ is selected from methylene, ethylene, propylene, butylene, pentylene, hexylene, heptylene, octylene, nonylene, decylene, undecylene and dodecylene.

41. The di-substituted pyridinium polymer composition of claim 34, wherein X is selected from a halide, triflate, mesylate and tosylate group.

42. The di-substituted pyridinium polymer composition of claim 41, wherein X is selected from bromide, chloride and iodide.

43. The di-substituted pyridinium polymer composition of claim 34 which is selected from cyclic and linear polymers.

44. A di-substituted pyridinium polymer selected from poly-(3-(12-bromododecyl) pyridine having 50 to 60 monomer units and a molecular weight of 12 to 15 kDa, and poly-(3-(8-bromooctyl)pyridine having 60 to 70 monomer units and a molecular weight of 11 to 13 kDa.

45. The di-substitued pyridinium polymer composition of claim 34 for a use selected from an antibacterial agent, an acetylcholine esterase inhibitor, a haemolytic agent, and a transfection reagent.
